(19) United States
(12) Patent Application Publication
(10) Pub. No.: US 2001/0049585 A1
(43) Pub. Date: Dec. 6, 2001

(54) Computer predictions of molecules

(76) Inventors: Garry Paul Gippert, Copenhagen (DK); Ole Lund, Copenhagen (DK); Thomas Nordahl Petersen, Copenhagen (DK); Claus Lundegaard, Bagsvaerd (DK); Morten Nielsen, Frederiksberg (DK); Soren Brunak, Hellerup (DK); Jakob Bohr, Humlebaek (DK); Henrik Bohr, Holte (DK)

Correspondence Address: BIRCH STEWART KOLASCH & BIRCH, PO BOX 747, FALLS CHURCH, VA, 22040-0747, US

(21) Appl. No.: 09/753,723

(22) Filed: Jan. 4, 2001

Related U.S. Application Data
(60) Provisional application No. 60/174,705, filed on Jan. 6, 2000.

(30) Foreign Application Priority Data
Jan. 5, 2000 (DK) PA 2000 00006

(51) Int. Cl. G06F 19/00; G01N 33/48; G01N 33/50

(52) U.S. Cl. 702/19

(57) ABSTRACT

A method for predicting a set of chemical, physical or biological features related to chemical substances or related to interactions of chemical substances including using at least 16 different individual prediction means, thereby providing an individual prediction of the set of features for each of the individual prediction means and predicting the set of features on the basis of combining the individual predictions, the combining being performed in such a manner that the combined prediction is more accurate on a test set than substantially any of the predictions of the individual prediction means.

The present invention relates in a first aspect to a method for prediction a set of chemical, physical or biological features related to chemical substances or related to interactions of chemical substances.

### BACKGROUND OF THE INVENTION AND INTRODUCTION TO THE INVENTION

The amount of data from the genome projects is increasing at rates difficult to manage by the modern scientist and current technologies. There is, thus, a need for useful means of extracting usable information from this data.

The protein-folding problem is one of the greatest unsolved problems in structural biology. The present invention seeks to extract information form the genome projects to advance the current understanding and to contribute to solving the protein-folding problem.

In 1963, Anfinsen demonstrated that denatured and thus unfolded proteins returned to their native structure once transferred to an appropriate medium, thus validating the theory that the secondary and tertiary structure of a protein is uniquely determined by its sequence of amino acids.

The present invention serves to calculate the structure and/or the structural, biological, chemical or physical features of chemical substances from their constituents, such as the features of proteins from their amino acid sequence. If the secondary structure or other features can be predicted with sufficient accuracy this could greatly enhance the homology based modelling of proteins and enable selection of molecules e.g. in drug discovery based on their inherent properties. Prediction of the secondary structure of proteins can be used to determine the tertiary structure of proteins by being used in the search for other proteins with similar secondary structures (fold recognition), or by being used to construct constraints that can help in the determination of the tertiary structure of a protein.

Neural networks have been used in related fields for a variety of purposes such as estimating binding energies (Braunheim, B. B., Miles, R. W., Schramm, V. L., Schwartz, S. D., Prediction of inhibitor binding free energies by quantum neural networks. Nucleoside analogues binding to trypanosomal nucleoside hydrolase. Biochemistry Dec. 7, 1999;38(49):16076-83), analyzing NMR spectra (Pons, J. L., Delsuc, M. A, RESCUE: an artificial neural network tool for the NMR spectral assignment of proteins. J Biomol NMR 1999 September;15(1):15-26), predicting the location of proteins (Schneider, G., How many potentially secreted proteins are contained in a bacterial genome? Gene Sep. 3, 1999;237(1):113-21), predicting O-glycosylation sites (Gupta, R., Jung, E., Gooley, A. A., Williams, K. L., Brunak, S., Hansen, J., Scanning the available Dictyostelium discoideum proteome for O-linked GIcNAc glycosylation sites using neural networks. Glycobiology 1999 October;9(10):1009-22), formula optimization (Takayama, K., Takahara, J., Fujikawa, M., Ichikawa, H., Nagai, T., Formula optimization based on artificial neural networks in transdermal drug delivery; J Controlled Release Nov 1, 1999;62(1-2):161-70f), and toxicity (Cai, C., Harrington, P. B., Prediction of substructure and toxicity of pesticides with temperature constrained cascade correlation network from low-resolution mass spectra; Anal. Chem. Oct 1, 1999;71(19):41, 34-41).

Overviews of different methods for making predictions for biological systems can be found in Durbin, R., Eddy, S., Krogh, A., Mitchison, G., Biological sequence analysis: Probabilistic models of proteins and nucleic acids, Cambridge University Press, Cambridge, UK, 1998 and in Brunak, B., Baldi, P., Bioinformatics: The Machine Learning Approach, MIT Press, Cambridge, Mass., 1998. The prediction of ab initio protein tertiary structure from the amino-acid sequence remains one of the biggest challenges in structural biology. One step toward solving this problem is by increasing the accuracy of secondary structure predictions for subsequent use as input to ab initio calculations or threading algorithms. Several studies have shown that an increased performance in secondary structure prediction can be obtained by combining several estimators (Rost, B., Sander, C., Prediction of protein secondary structure at better than 70% accuracy. J. Mol. Biol., 323:584-599 (1993); Cuff, J. A. & Barton, G. J. Evaluation and improvement of multiple sequence methods for protein secondary structure prediction. Proteins, 34:508-519 (1999)). A combination of up to eight neural networks has been shown to increase the accuracy, but a saturation point was reached in the sense that adding more networks would not increase the performance substantially (Chandonia, J. -M., & Karplus, M. New methods for accurate prediction of protein secondary structure. Proteins, 35:293-306 (1999)). Early methods for predicting protein secondary structure relied on the use of single protein sequences (Chou P. Y. and Fasman, G. D. Conformational parameters for amino acids in helical, sheet and random coil regions, calculated from proteins. Biochemistry, 13: 211-222 (1974); Garnier, J., Osguthorpe, D. J., and Robinson, B. Analysis and implications of simple methods for predicting the secondary structure of globular proteins. J. Mol. Biol 120: 97-120 (1978); Qian, N., Sejnowski, T. J., Predicting the secondary structure of globular proteins using neural network models, J. Mol. Biol., 202:865-84 (1988); Bohr, H., Bohr, J., Brunak, S., Cotterill, R. M., Lautrup, B., Norskov, L., Olsen, O. H, Petersen, S. B., Protein secondary structure and homology by neural networks. The alpha-helices in rhodopsin. FEBS Lett., 241:223-8 (1988)). Several groups have shown that a significant increase in performance can be obtained by using sequence profiles (Rost, B., Sander, C., Prediction of protein secondary structure at better than 70% accuracy. J. Mol. Biol., 323:584-599 (1993)) or position specific scoring matrices (Jones, D. T. Protein secondary structure prediction based on position-specific scoring matrices. J. Mol. Biol., 292: 195-202 (1999)).

The so-called PHD method developed by Rost and Sander was the method that performed best in the CASP2 experiment with a mean Q3 of 74% (Lesk, A. M. CASP2: report on ab initio predictions. Proteins. Suppl 1:151-66 (1997)). This method had a cross validated performance above 72% (Rost, B., Sander C. Combining evolutionary information and neural networks to predict protein secondary structure. Proteins, 19:55-72 (1994)). In a recent comparative study, the PHD method had the best Q3 (71.9%) of all individual methods tested, while a consensus method scored 72.9% (Cuff, J. A. & Barton, G. J. Evaluation and improvement of multiple sequence methods for protein secondary structure prediction. Proteins, 34:508-519 (1999)). In CASP3 the PSI-PRED method (Jones, D. T. Protein secondary structure prediction based on position-specific scoring matrices. J. Mol. Biol., 292: 195-202 (1999)) performed best with Q3 performances of 73.4% and 74.6%, respectively, on the two small test sets used by the evaluators. The PSI-PRED method was approximately seven percentage points better than a version of the PHD method similar to the one used in CASP2 (Orengo, C. A., Bray, J. E., Hubbard, T., LoConte, L., Sillitoe, I., Analysis and assessment of ab initio three-dimensional prediction, secondary structure, and contacts prediction. Proteins. Suppl 3:149-70 (1999)). In his paper, Jones reports a Q3 performance of 76.5% using a CASP-like secondary structure category definition, and a Q3 performance of 78.3% with a plain DSSP definition of secondary structure. The work done by the present inventors have resulted in a significant improvement over the Jones method as demonstrated by a Q3 performance of more than 80%.

An increased performance (Q3) in secondary structure prediction is known to be obtained by using a combination of a few predictions (Rost, B. & Sander, C., Prediction of protein secondary structure at better than 70% accuracy. J. Mol. Biol, 323:584-599 (1993); Cuff, J. A. & Barton G. J. Evaluation and improvement of multiple sequence methods for protein secondary structure prediction, Proteins, 34:508-519 (1999)).

In the articles by Riis and Krogh, 1996 (Riis S K, Krogh A. J. Improving prediction of protein secondary structure using structured neural networks and multiple sequence alignments. Comput Biol 1996 3:163-83.), and Riis, 1995 (Riis SK. Combining neural networks for protein secondary structure prediction. IEEE international conference of neural networks proceedings, (1995)), the authors use five networks for each of three different secondary structure types and these predictions are combined using another neural network. Furthermore, they use a local encoding scheme for the input and no encoding of the output is applied.

The article by Rost and Sander, 1993 (Rost B, Sander C. Improved prediction of protein secondary structure by use of sequence profiles and neural networks. Proc Natl Acad Sci U S A 90:7558-62 (1993)), describes the use a jury of networks that predicts by a simple vote of a set of 12 different networks. Also this method does not include encoding of the output.

Baldi et al., 1999 (Baldi P, Brunak S, Frasconi P, Soda G, Pollastri G. Exploiting the past and the future in protein secondary structure prediction. Bioinformatics 1999 15:937-46. (1999)), describe neural network architectures which do neither use combinations of prediction means nor encoding of the output.

In the article by Fumiyoshi, 1993 (Fumiyoshi S. Application of a neural network with a modular architecture to protein secondary structure prediction. Fujitsu-scientific and technical journal. 29:250-256, (1993)), the authors combine n-1 neural networks, to make a n state secondary structure prediction (n=3,4,8). The outputs from these neural networks are then combined in a unification unit.

A combination of up to eight neural networks has been shown to increase the accuracy (Chandonia, J. -M., & Karplus, M. New methods for accurate prediction of protein secondary structure, Proteins, 35:293-306 (1999)). Notably, these studies indicated that a saturation point had been reached in the sense that adding more networks would not increase the performance substantially.

According to the present invention, the performance obtained by using the prediction method and system disclosed herein is, surprisingly, dramatically better by combining up to 800 prediction means, beyond the so-called saturation point.

By the term prediction means we refer to a predictor preferably being, but not restricted to, a neural network. A prediction means such as a neural network may according to the present invention typically have many input units, typically one for each type of amino acid in each position of the input window. These input units are not regarded as independent prediction means but as different inputs to one prediction means.

Structure predictions have been performed by various methods including knowledge-based systems using statistical calculations from databases, sequence pattern recognition systems, methods based on physical or chemical properties of amino acids and neural networks.

A problem in connection with such methods is that the current level of accuracy is not sufficient to be able to reliably predict the secondary or tertiary structure from the amino acid sequence. Technical problems with the current neural network prediction systems, in that the number of networks through which the sequences are passed, as well as the diversity of these networks, the arrangement of the networks and most importantly the method by which the networks are averaged and the selection of networks is based on the available computer power leading to a selection of only the “best” networks (i.e. individual networks giving best predictions on a given test set).

### BRIEF DESCRIPTION OF THE INVENTION

This problem has been solved by means of the present invention which provides

in a first aspect a method for predicting a set of chemical, physical or biological features related to chemical substances or to chemical interactions using a system comprising a plurality of prediction means, the method comprising

using a plurality of different individual prediction means, such as at least 16, or such as at least 48, thereby providing an individual prediction of the set of features for each of the individual prediction means and

predicting the set of features on the basis of combining the individual predictions,

the combining being performed in such a manner that the combined prediction is more accurate on a test set than substantially any of the predictions of the individual prediction means.

In a second aspect, the invention relates to method for prediction of descriptors of protein structures or substructures comprising

feeding input data representing at least one residue of a protein sequence to at least 16 diverse neural networks arranged in parallel in a first level

generating by use of the networks arranged in the first level a single- or a multi-component output for each networks the single- or multi-component output representing a descriptor of one residue comprised in the protein sequence represented in the input data, or the single- or multi-component output representing a descriptor of 2 or more consecutive residues of the protein sequence

providing the single- or multi-component output from each network of the first level as input to one or more neural networks arranged in parallel to a subsequent level(s) in a hierarchical arrangement of levels, optionally inputting one or more subsets of the protein sequence and/or substantially all of the protein sequence to the second or subsequent level(s),

generating by use of the networks arranged in the subsequent level(s) single or multi-component output data representing a descriptor for each residue in the input sequence,

weighting the output data of each neural network of the subsequent level(s) to generate a weighted average for each component of the descriptor,

optionally selecting from the multi-component output data, if generated, the component of the descriptor with the highest weighted average as the predicted descriptor for each amino acid in the protein sequence, or optionally assigning a descriptor to a single-component output, and

optionally assigning the descriptor of the at least one residue of a protein sequence

In a third aspect, the invention provides a method for predicting a set of chemical, physical or biological features related to chemical substances or related to interactions of chemical substances

using a system comprising a prediction means comprising output expansion,

the method comprising

using at least 1 individual prediction means predicting substantially the whole set of features at least twice thereby providing at least two individual predictions of substantially all of the set of features, and

predicting the set of features either on the basis of

combining at least two of the individual predictions, the combining being performed in such a manner that the combined prediction is more accurate on a test set than substantially any of the at least two of the predictions, or

on the basis of selecting one of the sets of predictions, the selection being performed in such a manner that the selected prediction is more accurate on a test set than a prediction from corresponding prediction means without the use of output expansion,

or predicting the set of features on the basis of at least one individual predictions, or combining at least two of the individual predictions, the combining being performed in such a manner that the combined prediction is more accurate on a test set than substantially any of the predictions of the individual prediction means, or more accurate than corresponding prediction means not comprising output expansion.

A fourth aspect of the invention relates to a method of predicting a set of features of input data where the input data provided to a first level of neural networks is further inputted to the subsequent levels of neural networks.

Further aspects of the invention relate to prediction systems based on such methods and to methods for establishing a prediction system for predicting a set of chemical, physical or biological features related to chemical substances or to chemical interactions represented by an input data using a system comprising a plurality of prediction means, is provided by performing the steps according to any of the prior aspects of the present invention.

### DETAILED DESCRIPTION OF THE INVENTION

The use of the present invention serves to predict structural features with greater accuracy than current technologies by using massive averaging over many prediction means, such as neural networks, in which all or substantially all of the prediction means are included in the averaging has surprisingly given more accurate predictions than methods wherein so-called “stupid prediction means”, as judged by their prediction, are excluded.

In the present application, a number of terms are used which are commonly used in the prediction literature. An explanation of some of the special terms and concepts relevant to the present invention is given in the following items:

Accurate:

in itself or when applied to the terms prediction, as in claim 1, is intended to mean a prediction more similar to the correct prediction, on a given data set, using a given measure of similarity. The accuracy is the similarity between the predicted output and the correct output, given a measure of similarity. The correct output is the output that the person constructing the predictor wants the predictor to give. The correct output may be extracted from experimental data, such as results from X-ray or NMR experiments. The measure of similarity may, for example, be the percentage of outputs, such as the number of prediction in a series of predictions, where the predicted output identical to the correct output is divided by the total number of outputs, multiplied by 100. Without being limited to a particular method, the measure of similarity may alternatively be the number of correct predictions, that is to say the number of examples in the test set where the predicted output is identical to the correct output.

Learning rate:

The parameter in the neural network proportional to the change in weights in the neural network which occurs during training of a neural network. A feature-specific learning rate may be constant or a function of the data set. It may be vary such that it is larger for some subtypes of output data (e.g. larger for helix than for coil), or on subsets of the data (e.g. larger on some sequences than on others).

Type (or types):

When applied to prediction means the term include but are not limited to neural networks, hidden Markov models (HMMs), EM algorithms, weight matrices, decision trees, fuzzy logic, dynamical programming, nearest neighbour approaches, Gibbs sampling and vector support machines as well as others known by the person skilled in the art.

Architecture:

When applied to the term prediction means or neural network the term is intended to mean the organisation of parameters in a prediction means or neural network including the number connectivity of units, the number of window sizes, the size of a window, and/or the number of hidden units. In neural networks, it may further refer to the number of neurons in different layers of neurons and/or the connections between these. When applied to HMMs, the term architecture may further refer to the definition of states and the connectivity of states. The parameters of an architecture are well known to the person skilled in the art.

Prediction means:

A prediction mean is a system capable of giving a prediction. A prediction mean may also be defined as a specification for how to calculate an output. The output from a prediction mean is called a prediction. This calculation may or may not depend on data given to the method as input.

A prediction mean may consist of other prediction means. They can be arranged in levels so that the output from one layer is used as input to the next layer. Each level may consist of one or more prediction means.

Prediction means may be different, i.e. different prediction means, in the way that an output is calculated and/or different in the parameters used to calculate the output. These differences may arise from using different input to the prediction mean, constructing it to give a different output, giving the prediction mean a different architecture, or training it on different data sets.

Functionally they may be different in that they can give a different output, even if they are given the same input.

Prediction means may be diverse with respect to type, and/or with respect to architecture, and/or in case of prediction means subjected to training with respect to initial conditions, and/or with respect to training thereby providing prediction means that may be capable of giving an individual prediction different from the individual prediction given by any of the other prediction means for at least one set of input data.

Prediction or predictions:

Is intended to mean an output by a prediction means. An individual prediction is intended to mean the output for a single residue or element in a sequence. Said sequence has as an output a series comprising a plurality of individual predictions.

Descriptor or descriptors:

Is intended to mean the chemical, physical or biological features related to chemical substances or to chemical interactions of molecules or subsets of molecules to be predicted by means of output data by a prediction means or comprised in the output data in a training set. Descriptors may be selected from the group comprising secondary structure class assignment, such as helix, extended strand, coil and/or p-sheet, tertiary structure, interatomic distance, bond strength, bond angle, descriptors relating to or reflecting hydrophobicity, hydrophilicity, acidity, basicity, relative nucleophilicity, relative electrophilicity, electron density or rotational freedom, scalar products of atomic vectors, cross products of atomic vectors, angles between atomic vectors, triple scalar products between atomic vectors, torsion angles, atomic angles such as but not exclusively omega, psi, phi, chi1, chi2, chi21, chi3, chi4, chi5 angles, chain curvature, chain torsion angles, and mathematical functions thereof.

Input data:

Input data is the data fed to the prediction means. In the training mode, input data further comprises the features that may be predicted by the prediction means. The sub-type of input data may be selected from the group comprising sequence profile, amino acid composition, amino acid position, windows of amino acids, peptide length and descriptors. Input data may comprise a number of elements each comprising one or more corresponding features. The input data may for example comprise one or a plurality of amino acid sequences. Each element may be an amino acid in a protein sequence. The feature of each element may be the secondary structure of that amino acid. Each feature may be described by a single or a plurality of descriptors. The feature secondary structure, for example, may be defined using from about 1 to 10 descriptors, such as alpha-helix.

Window size:

Window size is the number of elements or residues within a sequence of elements or residues. The term window is intended to mean the sequence of elements or residues.

Output data:

Output data is intended to mean data generated by use of the prediction mean and may comprise of a descriptor or any chemical, physical or biological feature related to chemical substance or to chemical, physical or biological interactions of molecules or subsets of molecules. Subtypes of output data are of one or more subtypes of input data used in the training mode. A subtype of output data may be selected from the group comprising sequence profile, amino acid composition, amino acid position, windows of amino acids, peptide length and descriptors.

Output expansion:

Output expansion is intended to mean the process by which the single- or multi-component output represents the features of 2 or more input elements. Substantially all of the elements will therefore have their features predicted at least twice. One or more of these at least two predictions may be more accurate than a corresponding prediction without output expansion, or a prediction based on a combination of at least two of these predictions may be more accurate than a prediction without output expansion. In an preferred embodiment, the features of 2 or more residues refers to the features of consecutive residues in a sequence, such as in a protein sequence.

Sequence profile:

Sequence profile is intended to mean the position specific probability of finding a given amino acid on a given position in a multiple alignment of related sequences. From the stacked sequences generated upon alignment of the sequences a position specific scoring matrix or log-odds scoring matrix may also be generated.

Training set:

Training set is intended to mean the input data used to train a prediction means. The training process may comprise feeding input data to a first level of prediction means, optionally feeding output data from the first level and/or input data previously fed or not fed into the previous level to a subsequent level or levels, an output expansion, a weighting of components of output data and a cross-validation process. The training of a neural network means using a training example to adjust the parameters in the neural network. A training set may comprise of all or part of the input data. Input data may be conceptually and practically divided into a training set and a test set. The training set is used to adjust the weights of the neural network and the test set is used to evaluate how accurate the neural network can predict. Testing of a neural network means using a test set to evaluate how accurate a neural network, preferably a network that previously underwent training, can predict. The training of a network involves performing a number of training cycles using a training set. At each training cycle, all input data or a subset of the input data from the training set is used as input to the neural network. On the basis thereof, the neural network produces a predicted output. The predicted output is compared to the correct output, and the weights of the neural network is adjusted, preferably using the back propagation algorithm, typically with the aim of reducing the difference between the predicted output and the correct output. The weights may be adjusted after each training example has been presented to the neural network (on line training), or after all training examples have been presented to the neural network (off line training). After the training cycle, a test cycle may be performed, preferentially after each training cycle. In a test cycle, input data from the test set and/or corresponding feature or features is fed to the neural network, the predicted output is calculated, and it is compared to the correct output. The accuracy of the predictions on the test set may be calculated. A plurality of training cycles may be performed. The number of training cycles to be performed may be fixed before, during or after the training starts. The weights used for the subsequent predictions or queries may be selected as the weights after the last training cycle, or preferably as the weights from the cycle which gave the best accuracy on the test set.

The accuracy of neural networks may be established by using a data set which has neither been used to train nor to test the accuracy of a neural network called an evaluation set. The evaluation set may also be used to test the accuracy of combinations of neural networks either in a single level or in multiple levels.

Cross-validation procedure:

Cross validation procedure is a process wherein X-Y subsets of training sets (wherein X≧Y) of X input data are used to train a prediction means and Y is the number of subsets of test sets. Preferably, in the cross validation procedure, the data set is divided into X subsets and the network is trained on X-1 of the subsets called the training set and tested on the last subset called the test set as. This may be done X times on each prediction means, each time using a different subset as the test set.

Diversity (or its corresponding diverse):

When applied to neural networks diverse are intended to mean networks which are diverse with respect to architecture and/or initial conditions and/or selection of learning set, and/or position-specific learning rate, and/or subtypes of input data presented to respective neural networks, and/or the randomisation of weights, and/or with respect to subtypes of output data sets rendered by the respective neural networks.

Weighting (or its corresponding weighted average):

An output produced by the selected prediction means may be a single-component such as a scalar or multi-component such as a number of scalars ordered for instance in a vector. In general the weighting comprises multiplication of each component of a single- or multi-component output for each residue by a weight, said weight being a per-sequence estimated performance obtained for the chain and prediction means in question. The resulting products are summed for each residue and component, and the resulting sums are divided by the sum of weights. Finally, the resulting maximal per-residue component quotient is used to determine the descriptor of the residue in question, and the per-sequence per-prediction probability of the descriptor is averaged over a given protein chain.

Per-residue-confidence rating, per-chain-confidence rating, and per-subset-of-chain-confidence rating:

These terms are intended to mean the score of the weighting process for each residue, chain, or subset of chain, respectively.

Initial conditions:

Is intended to mean the conditions to which a prediction means are set prior to performing a prediction and include architecture, training set, learning rate, weighting process, subtype of input data, and input data.

According to the first aspect of the present invention, at least 16 different individual prediction means are applied which may be selected from a plurality of prediction means, which plurality may comprise more than 16 prediction means. Each of the 16 different individual prediction means predicts individually a set of features where after the prediction predicted by the method is provided by combining the individual prediction means. In a preferred embodiment of the method according to the invention the combining being performed is an averaging and/or weighted averaging process. The averaging applied may be a mean value obtained by summing up the prediction and dividing by the number of prediction and the weighted averaging may preferably be constituted by multiplying each prediction by a number followed summation of the multiplied predictions and dividing by the number of predictions. Furthermore, a combination of these two measures may be applied in which case a fraction of the predictions are multiplied and the remaining predictions are use as they are.

The combining of the predictions provided by the individual prediction means are based on predictions provided by either substantially all or all prediction means of the system or substantially all or all prediction means of the system which do not compromise the accuracy of the combined prediction or substantially all or all prediction means of the system which are accurate above a given value or substantially all or all prediction means of the system which are estimated to be accurate above a given confidence rating.

Typically, the combining of the predictions provided by the individual prediction means are based on predictions provided by either substantially all or all prediction means of the system or substantially all or all prediction means of the system which do not compromise the accuracy of the combined prediction or substantially all or all prediction means of the system which are accurate above a given value or substantially all or all prediction means of the system which are estimated to be accurate above a given confidence rating.

The term substantially all of the prediction means implies that it is not always essential for all of the prediction means to be utilised for combining. In some embodiments, substantially all implies that at least 50% of the prediction means are used, whereas in other embodiments, at least 75% of the prediction means are used such as at least 80%, 90% or 95% are used.

The selection or deselection of individual prediction means may be based on the “accurate above a given value” which may be calculated during the development of the prediction means. Alternatively, the selection process may be based on the estimated accuracy during a prediction of a blind test set, that is to say where the correct prediction is not known.

In preferred embodiments of the present invention the value above which a prediction is considered to be accurate is such that the individual prediction means in question is selected if it does not raise the standard deviation of the prediction accuracies by more than 500%, such by not more than 200%, such as 100% or 50% or it is deselected if its accuracy is a number of standard deviations below the average accuracy.

The number of different predictions means may be at least 16, such as at least 20, such as at least 30, such as at least 40, 50, 75, 100, 200, 400, 600, 800, 1000, 1200, 1400, 1600, 1800, 2000, 2500, 3000, 4000, 5000, 6000, 7000, 8000, 9000, 10,000, 15,000, 20,000, 30,000, 40,000, 50,000, 100,000, 200,000, 500,000, 1,000,000. The actual number of prediction means may vary depending on the prediction problem and may be determined empirically during the development of the prediction system for the individual prediction problem.

Typically, types of prediction means are selected from the group consisting of neural networks, hidden Markov models (HMMs), EM algorithms, weight matrices, decision trees, fuzzy logic, dynamical programming, nearest neighbour approaches, and vector support machines. It is equally anticipated that the prediction means may comprise a combination of different types of prediction means, such as combining neural networks with HMMs or dynamical programming.

In preferred embodiments, the prediction means may be diverse with respect to type, and/or with respect to architecture, and/or in case of prediction means subjected to training with respect to initial conditions, and/or with respect to training thereby providing prediction means that may be capable of giving an individual prediction different from the individual prediction given by any of the other prediction means for at least one set of input data.

As stated the prediction system comprises a combining of individual predictions, preferably where the combining is a weighted averaging process. This weighted averaging process may be performed based on the accuracy of substantially each or each of the individual prediction means. The accuracy may be an estimated accuracy, or a measured accuracy on a test set or a combination of those.

In certain embodiments, a sequence of the individual predictions performed is a series of predictions, and the weighting comprises an evaluation of the relative accuracy of substantially each individual prediction or each individual prediction means on substantially all, or one or more subsets of the predictions in a series of predictions.

A series of predictions is a plurality of predictions possessing a connectivity such as a physical, logical, or conceptual connectivity.

In a preferred embodiment of the invention, the method for prediction is applied in an adaptive way. The adaptivity may preferably be established by the weighting of particular individual predictions resulting in an evaluation that the predictions rendered by the systems on substantially all or one or more of the subsets of the predictions in a series of predictions are to be excluded from the weighted average and/or the individual prediction means in question may be excluded from the weighted average in further predictions, either with respect to substantially all or with respect to one or more of the subsets of the predictions in a series of prediction.

The number of prediction means evaluated not excluded from the weighted average and/or the individual prediction means not excluded from the weighted average in further predictions is preferably at least 3 such as 4, preferably at least 5, 6, 7, 8, 9, or 10.

The confidence rating is preferably calculated by multiplying each component of an individual prediction of the selected prediction means

by the weight obtained for a sequence and prediction means,

the resulting product summed for each component of each residue over all prediction means,

the resulting sums being divided by the sum of weights, and

the resulting maximal per-residue component quotient being used to determine the H or E or C secondary structure assignment for that residue.

Optionally, further to such assignment, the estimated accuracy of the combined prediction can be calculated as the average maximal per-residue component quotient for the residues of the chain in question.

In preferred embodiments, the output of one level of prediction means comprises a descriptor of 2, 3, 4, 5, 6, 7, 8 or 9 consecutive residues, preferably 3, 5, 7, or 9 consecutive residues.

The invention relates to predicting a set of features of an input data by providing said input data to at least 16 diverse neural networks thereby providing an individual prediction of the said set of features on the basis of a weighted average said weighted average comprising an evaluation of the estimation of the prediction accuracy for a protein chain by a prediction means.

Another aspect of the invention relates to a method for predicting a set of features of input data using output expansion wherein a process by which a single- or multi-component output is represented by a descriptor of 2 or more consecutive elements of a sequence, such as residues of a protein sequence.

In preferred embodiments, output expansion is used alone or in combination with the prediction system disclosed herein. As stated, one aspect of the invention relates to a method for predicting a set of chemical, physical or biological features related to chemical substances or related to interactions of chemical substances using a system comprising a prediction means comprising output expansion, the method comprising using at least 1 individual prediction means predicting substantially the whole set of features at least twice thereby providing at least two individual predictions of substantially all of the set of features, and predicting the set of features either on the basis of combining at least two of the individual predictions, the combining being performed in such a manner that the combined prediction is more accurate on a test set than substantially any of the at least two of the predictions, or on the basis of selecting one of the sets of predictions, the selection being performed in such a manner that the selected prediction is more accurate on a test set than a prediction from corresponding prediction means without the use of output expansion, or predicting the set of features on the basis of at least one individual prediction, or on the basis of combining at least two of the individual predictions, the combining being performed in such a manner that the combined prediction is more accurate on a test set than substantially any of the predictions of the individual prediction means, or more accurate than corresponding prediction means not comprising output expansion.

It is to be noted that the primary reason the method comprises predicting only substantially the whole set of features at least twice thereby providing at least two individual predictions of substantially all of the set of features and not the whole set of features is merely a consequence of the obvious fact that all sequences terminate and thus, in output expansion, in which the features of residues are preferably features of neighbouring or consecutive residues, the terminal residues are not neighboured by more than one residue.

Furthermore, the invention relates to a prediction system established by said methods and/or a prediction system established by providing a system being able to perform said steps and/or a prediction system comprising a combination of systems established by said method or comprising a combination of systems established by said method and another type of system.

The number of prediction means averaged in the method described infra for predicting the chemical, physical, or biological features of chemical substances or for predicting said features related to interactions of chemical substances is unprecedented in such types of prediction systems.

Furthermore, a prediction system wherein in addition to the at least one subtype input data fed into a first level of prediction means (referred to as a sequence-to-structure level) is also fed into at least one subsequent level of prediction means, at least one subtype of data provided by the first level or prior level of prediction means is fed changed or unchanged to at least one subsequent level (a structure-to-structure level) is significantly more accurate than systems wherein no such structure-to-structure level of prediction means is run in addition to a sequence-to-structure level of prediction means. Preferred embodiments comprise at least one sequence-to-structure level and at least one structure-to-structure level.

Moreover, a prediction system comprising output expansion was surprisingly found to be more accurate than one without output expansion.

One aspect of the invention comprises, in general terms, the establishment of a prediction system by training of a number of differing prediction means by providing input data whose output data is known. The training is tested and cross-validated for each of the prediction means. For a query, the input data is fed into the each of the trained prediction means and a mass averaged prediction is made from each of the output data.

In general, the input data and/or its features have a corresponding or complementary output data. Moreover, the input elements can be arranged in one or more sequences, such as amino acid residue or nucleic acid residue in a peptide or nucleotide, and that for each input element, predictions are made for more than one output element.

Furthermore, the more than one output elements correspond to neighbouring input elements.

Features and Descriptors

The features to be predicted by the system are descriptors of molecules or subsets of molecules. A molecule can have many features and hence many descriptors. Given that a seemingly simple molecule like water has features such as bond angles, bond lengths, rotation, hydrophilicity, acidity, basicity, polarity, and numerable vectors and scalar products, larger and more complex molecules may have these features and a multitude of others. As is known by the person skilled in the art, innumerable descriptors can be assigned to a chemical substance or to a portion or subset of the molecule.

In embodiments where a descriptor is to be predicted and assigned to a chemical interaction between two or more chemical substances, the nucleophilicity and/or electrophilicity of the chemical substances and/or moieties of the chemical substances can be particularly important. Moreover, their size and/or size of a pocket within the molecule, as well as polarity, hydrophobicity may be important. Relative bond strengths may also be of relevance. Given the number of vectors and scalar components involved in chemical interactions, as well as critical scalar and vector products, the person skilled in the art will appreciate the plurality of potential descriptors relevant in such interactions and to molecules in general.

In general, descriptors may be selected from the group comprising secondary structure class assignment, tertiary structure, interatomic distance, bond strength, bond angle, descriptors relating to or reflecting hydrophobicity, hydrophilicity, acidity, basicity, relative nucleophilicity, relative electrophilicity, polarity electron density or rotational freedom, scalar products of atomic vectors, cross products of atomic vectors, angles between atomic vectors, triple scalar products between atomic vectors, torsion angles, atomic angles such as but not exclusively omega, psi, phi, chi1, chi2, chi21, chi3, chi4, chi5 angles, chain curvature, chain torsion angles, and mathematical functions thereof.

The chemical, physical or biological features related to chemical substances or to chemical interactions to be predicted are typically descriptors of molecules or subsets of molecules.

In some embodiments, the descriptors are ascribed to features of molecules themselves whereas in others, they are ascribable to the interaction between molecules. Interacting molecules may be organic substances, inorganic substances, or the interaction may be an interaction between an inorganic and organic substance.

The organic substance may be protein, polypeptide, oligopeptide, protein analogue, peptidomimietic, peptide isostere, pseudopeptide, nucleotide and derivatives thereof, PNA and nucleic acids, or any compound used as for therapeutic, pharmaceutical, or diagnostic purposes. In one embodiment of the method, the interacting molecules are a receptor and a molecule able to bind to said receptor such as a metal, an antagonist or agonist. In another embodiment, the molecule or interaction under investigation is organometallic or a metal-organic complex.

In preferred embodiments, the molecules are selected from the group comprising proteins, peptides, polypeptides and oligopeptides. These may be metalloproteins or purely organic in nature. The proteins, polypeptides or oligopeptides may also be self-complexed, complexed with another type of organic molecule or complexed with an inorganic compound or element.

Data

The features and/or descriptors may be a subtype of data fed into the prediction means. Further subtypes of data may comprise amino acid sequence, nucleotide sequence, sequence profiles, windows, amino acid composition, nucleic acid composition, length of protein or length of protein and descriptor.

From the data set, a plurality of corresponding input and output examples may be constructed. If the data set is one or more amino acid sequences and their corresponding secondary structures, an input example may consist of a window of amino acids surrounding a central amino acid and the output example may consist of the secondary structure corresponding to the central amino acid. In this way corresponding input-output examples may be constructed for each amino acid in the data set.

The invention and, in particular, different aspects and embodiments thereof, may be further described in relation to articles or in relation to prior art. References are made where appropriate to articles giving the background of the invention. It is to be emphasised that the scope of the invention should not be construed in a limiting sense in the cases where references to prior art are made.

The data may be raw or may be filtered prior to being fed to the prediction means. In one embodiment of the invention, the raw data may come from a commercial or publicly available data bank such as a protein data bank. The input data may be unchanged or, upon filtration through one or more quality filters, may be taken from a biological or chemical database, such as a protein database, a DNA data base and an RNA database.

In preferred embodiments, the data is passed through one or more filters. In one such embodiment, the raw data may be passed sequentially through three filters for i) structure quality check, ii) homology reduction, and iii) manual reduction. A second round of homology reduction may also take place.

In embodiments where the raw data is obtained from a protein data bank, the structure filter quality filter (pdf2pef program) may exclude protein chains if

(1) Secondary structure could not be assigned by the program DSSP (Kabsch and Sander, 1983)

(2) Occurrence of chain breaks (defined as consecutive amino acids having C-α-distances exceeding 4.0 Å)

(3) X-ray structure solved to a resolution worse 2.5 Å

(5) DSSP length <30 (units) (Kabsch, W. and Sander, C. A dictionary of protein secondary structure. Biopolymers. 22: 2577-2637 (1983))

(6) Fraction of coil (dot)>0.5

(7) Fraction of E+H<0.2.

Variable parts NMR chains may be excluded if:

(4) Multiple NMR chains superimposed with a distance r.m.s>1 Å, determined using the program domain.

In the homology reduction filter process, a representative set with low pairwise sequence similarity may be selected by running algorithm #1 of Hobohm (Hobohm, U. and Scharf, M. and Schneider, R. and Sander, C. Selection of a representative set of structures from the Brookhaven Protein Data Bank. Protein Sci. 1: 409-417 (1992)). The sequences may be aligned using the local alignment program, ssearch© (Myers, 1988; Pearson, 1990) using the pam 120 amino acid substitution matrix (Dayhoff, M. O., Schwartz, R. M., Orcutt, B. C. A model of evolutionary change in proteins. Atlas of Protein Sequence and Structure, 5, Suppl. 3: 345-352 (1978)), with gap penalties −12, −4. A cutoff for sequence similarity may be calculated by I_{—}=290/sqrt(L), where I is the percentage of identical residues in the alignment and L is the length of the alignment.

In general, in a manual filtration process, one may visually examine the data set and remove any data set at random or manually selectively removed for reasons specific to the query. In the manual filter process, in embodiments where descriptors of a protein sequence are to be predicted, the trans-membrane and integral-membrane proteins may be removed. Also, in certain instances, non-globular proteins may be removed from the data set.

Preferably, a second round of homology filtration may take place.

Optionally, second and subsequent filtration processes of each type of filtration process may be performed.

In the preferred embodiment where a second round of homology filtering takes place, sequences from the manual filtration having a sequence similarity above the previously defined threshold to the set of 126 sequences used by Rost and Sander (1993) were removed. This data set is referred to as the TT set.

The TT set may be employed for statistical examination and prediction algorithm developments and other sets such as the 126 sequences used by Rost and Sander (1993) (the RS set) may be used as an independent validation set. The TT set of protein chains may be divided randomly into sub sets, such as 10 subsets assigned as TT1-TT10.

In the preferred embodiment where a feature, such as a secondary structure, is used as input data, the secondary structure may be assigned to the input data. In one non-limiting embodiment, the DSSP program (Kabsch and Sander, 1983) may be used to assign features to input data wherein eight different DSSP secondary structure classes {H,G,I,E,B,T,S, .} may be merged into a three state assignment by the rules: H is converted into helix (H), E is converted into strand (E), and the six others (G,I,B,T,S, .) are converted into coil (C).

Other methods of groupings may alternatively be used to assign the secondary structure. For instance, H and G may be converted to H; E and B may be converted to E; and the remaining may be converted to C.

Other programs may be used in conjunction with the DSSP program or may serve independently to assign features to input data. Accordingly, other programs may be used to assign the secondary structure or any other feature or descriptor.

Descriptors are typically selected from the group comprising secondary structure class assignment, tertiary structure, interatomic distance, bond strength, bond angle, descriptors relating to or reflecting hydrophobicity, hydrophilicity, acidity, basicity, relative nucleophilicity, relative electrophilicity, electron density or rotational freedom, scalar products of atomic vectors, cross products of atomic vectors, angles between atomic vectors, triple scalar products between atomic vectors, torsion angles, atomic angles such as but not exclusively omega, psi, phi, chi1, chi2, chi21, chi3, chi4, chi5 angles, chain curvature, chain torsion angles, torsion vectors and mathematical functions thereof.

Conformational parameters for amino acids in helical, sheet and random coil regions, calculated from proteins may be obtained by Chou, P. Y. and Fasman, G. D. (Biochemistry, 13: 211-222 (1974)).

In the embodiment where the input data comprises a sequence of element or residues, such as nucleotide sequence or a sequence of amino acid residues, the sequence profiles may be computed by running the program blastpgp from the psi-blast package 6.03 (Altschul, 1991) with the -j3 option (three iterations), and extracting the precision-specific scoring matrix produced by the program or the log-odds matrix from the output. If the blastpgp does not output any matrix, the sequence profile may be constructed from a blosum62 matrix (Henikoff, S. and Henikoff, J. G, Amino acid substitution matrices from protein blocks. Natl. Acad. Sci. U.S. A. 89: 10915-10919 (1992)). Alternatively, many other methods of computing the sequence profiles are anticipated.

Without being limited to a specific mode, the preparation of the sequence profiles may be done by a procedure in which the database sequences are preprocessed. Sequences are read from the latest version of the non redundant Swiss Prot+Trembl database (Bairoch, A. and Apweiler, R. The SWISS-PROT protein sequence data bank and its new supplement TREMBL. Nucleic Acids Res, 24: 21-25 (1996)). Sequence stretches where the feature table match FT SIGNAL, FT TRANSMEM, or FT DOMAIN with RICH|COIL|REPEAT|HYDROPHOBIC in the description are replaced with X's.

Prediction Means

The number of different predictions means used by the method is preferably at least 20, such as at least 30, such as at least 40, 50, 75, 100, 200, 400, 600, 800, 1000, 1200, 1400, 1600, 1800, 2000, 2500, 3000, 4000, 5000, 6000, 7000, 8000, 9000, 10,000, 15,000, 20,000, 30,000, 40,000, 50,000, 100,000, 200,000, 500,000, 1,000,000.

In one preferred embodiment of the invention, the number of prediction means is at least 48. The use of at least 48 prediction means may be used in, amongst others, aspects of the invention for predicting a set of chemical, physical or biological features related to chemical substances or related to interaction of chemical substances or for the prediction of descriptors of protein structures or substructures or for predicting a set of features of an input data by providing said input data to said prediction means.

Depending on the subtype of the input data and the type of prediction means as well as other variables such as the prediction problem itself, the number of prediction means required for a notable improvement in the accuracy of the prediction by means of the method described infra may vary. In some embodiments, the use of, for example, 20 000 prediction means may not provide a notable improvement over the use of 200 prediction means, whereas in other embodiments, where for example the subtype of input data or feature is different than the aforementioned example, the use of 1000 prediction means provides a notable improvement over the use of 20 prediction means. Preferably, for secondary structure prediction using neural networks as the prediction means, at least 800 neural network combinations may be used.

Possible embodiments comprise the use of prediction means selected from the group comprising neural networks, hidden Markov models (HMM), EM algorithms, weight matrices, decision trees, fuzzy logic, dynamical programming, nearest neighbour approaches, and vector support machines, preferably wherein the prediction means are neural networks.

Especially preferred embodiments of the method comprise an arrangement of predictions means, such as neural networks into at least two levels.

Generally, the number of neural networks in the one of the subsequent level or levels range from 1 to 1 000 000, such as from 1 to 100 000, 1 to 50 000, 1 to 10 000, 1 to 5000, 1 to 2500, 1 to 1000, 1 to 500, 1 to 250, 1 to 100, 1 to 50, 1 to 25 or 1 to 10.

In preferred embodiments, the output of one level of prediction means comprises a descriptor of 2, 3, 4, 5, 6, 7, 8 or 9 consecutive residues, preferably 3, 5, 7, or 9 consecutive residues.

Preferably, the prediction means of the system are arranged in levels and wherein at least one subtype of data provided by a first level of prediction means is transferred changed or unchanged to at least one subsequent level.

Single- or multi-component output (described infra) from at least one neural networks in at least one level in a hierarchical arrangement of levels of neural networks is preferably supplied as input to more than one neural network in a subsequent level of neural networks.

In one particularly attractive embodiment of the method, at least one subtype of data provided by a first level of prediction means is transferred changed or unchanged to at least one subsequent level, and at least one subtype of data provided to a first level of prediction means is also transferred changed or unchanged to at least one subsequent level.

Moreover, it may be preferable that the at least one subtype of data transferred to the at least one subsequent level comprises subsets of predictions provided by the first level of prediction means and/or subtypes of input data either changed or unchanged from input data fed into the first prediction means.

The prediction means may be different from one another with respect to type, and/or with respect to architecture, including differing in the number connectivity of units and/or window size, and/or randomisation of the initial weights and/or the number of hidden units.

Diverse networks may be diverse with respect to architecture and/or initial conditions and/or selection of learning set, and/or position-specific learning rate, and/or subtypes of input data presented to respective neural networks, and or with respect to subtypes of output data sets rendered by the respective neural networks.

Furthermore, the networks diverse in architecture may have differing window size and/or number of hidden units and/or number of output neurons.

The said sub-types of input data may be selected from the group comprising sequence profiles, amino acid composition, amino acid position and peptide length.

In one preferred embodiment, where the prediction means is a neural network and the input data is a sequence, four different window sizes and two different numbers of hidden units are used, such as 50 and 25, resulting in eight different network architectures. The window sizes may be any integer of at least one. Preferred window sizes may depend on the length of the sequence, the length of the subsequence or on any portion of the sequence that may have an influence on the feature to be predicted such as the secondary or tertiary structure. Preferably, at least one level in a hierarchical arrangement of levels of parallel neural networks comprises networks with at least 7, such as at least 9, such as at least 11, particularly at least an 11 residue input window, such as at least 13, 15, 17, 21, 31, 41, 51, or 101 residue input window. For a protein sequence, preferred embodiments of window sizes are at least 7, such as at least 9, such as at least 11, particularly at least 13, 15, 17, 19, 21, 23, 25, 27, 29, 31, 41, 51, 61, 71, 81, 91, or 101 residue input window.

Furthermore, at least one level in a hierarchical arrangement of levels of parallel neural networks comprises networks preferably have at least two different window sizes, such at least 3, 4, 5 or 6 window sizes.

Moreover, in at least one level in a hierarchical arrangement of levels of parallel neural networks comprises networks with at least 1 hidden unit, such as at least 2, 5, 10, 20, 30, 40, 50, 60, 75 or 100 hidden units.

In the preferred embodiments where the prediction means subjected to training, the predictions means may further differ with respect to initial conditions, and/or with respect to training including differing in architecture, training set, learning rate, weighting process, subtype of input data and/or input data.

Networks differing in their initial conditions may be selected by the process of randomly setting each weight to ±0.1 and/or randomly selected from [−1; 1].

Within one level of prediction means, the prediction means may differ from one another with respect to type. In certain embodiments, a level of prediction means may be different with respect to type to a subsequent level of prediction means. In preferred embodiments, the prediction means are of the same type such as all being neural networks or all being hidden Markov models (HMM), or all being EM algorithms, most preferably all being neural networks.

Prediction means within a level or from in a subsequent level may be different in that substantially each or each of the prediction means are of different type and/or will be capable of giving an individual prediction different from the individual prediction given by any of the other prediction means for at least one set of input data and/or has different initial conditions and/or has different architecture.

In embodiments where the prediction means are neural networks, the neural networks are diverse with respect to architecture, and/or with respect to initial conditions, and/or with respect to selection of training set, and/or with respect to learning rate.

In preferred embodiments, prediction means within a level and within a system are not different with respect to type, in that all the prediction means are neural networks, and are not different with respect to subtype of input data, in that all are fed a oligonucleotide, oligopeptide, polypeptide or protein sequence optionally with corresponding features, and are different with respect to subtype of output data rendered by the respective neural networks, in that all predict chemical, physical or biological features related to chemical substances or related to interactions of chemical substances, most preferably descriptors of secondary structures.

Preferably, the networks in a subsequent level are fed the predictions from networks in the first level or previous level as input, or as part of their input. The networks within these subsequent levels are therefore preferably trained after that the networks in the first or previous level have been trained. Using cross validation as described infra, one prediction is made for each of the X test sets, and these predictions may be chosen to be the data set for training the networks in the subsequent level. Additional information other than predictions from the first or prior level of networks may be fed into the networks in the subsequent level, such as the window of the sequence surrounding the amino acid for which the descriptor, such as the secondary structure, is to be predicted may given as additional input to the network or networks in the subsequent levels.

The prediction means are trained by a training process comprising an X-fold cross-validation procedure wherein X-Y subsets of training sets (wherein X≧Y) of X input data are used to train a prediction means and Y is the number of subsets of test sets. Preferably, in the cross validation procedure, the data set is divided into X subsets and the network is trained on X-1 of the subsets called the training set and tested on the last subset called the test set as. This may be done X times on each prediction means, each time using a different subset as the test set. In preferred embodiments, the prediction means are trained by a training process comprising an X-fold cross-validation procedure wherein each network was trained on (X-1) of X subsets of data and tested on 1 or more of said subsets. The term X may be any integer ranging from 2 to 1 000 0000, such as from 2 to 100 000, 2 to 10 000, 2 to 1000, 2 to 100, 2 to 50, preferably 5 to 50, such as 5, 10, 15, 20, 25, 30, 35, 40, 45 or 50. Preferable embodiments of this aspect of the cross-validation process comprise a 10-fold cross-validation process, i.e. where X is 10, and most preferably where Y is 1.

The testing on the subset comprises making a prediction for each element in the data set and evaluating the accuracy of the prediction.

The training process typically comprises i) supplying input data, filtered or unfiltered from a database, ii) generating by use of the networks arranged in the first level a single- or a multi-component output for each networks, the single- or multi-component output representing a descriptor of one residue comprised in the protein sequence represented in the input data, or the single- or multi-component output represents a descriptor of 2 or more, consecutive residues of a protein sequence, iii) providing the single- or multi-component output from each network of the first level as input to one or more neural networks arranged in parallel in a subsequent level(s) in a hierarchical arrangement of levels, iii) optionally inputting one or more subsets of the protein sequence and/or substantially all of the protein sequence to the subsequent level(s), iv) generating by use of the networks arranged in the second or subsequent level(s) a single or multi-component output representing a descriptor for each residue in the input sequence, v) weighting the output of each neural network of the subsequent level(s) to generate a weighted average for each component of the descriptor, and vi) performing an X-fold cross-validation procedure wherein each network was trained on (X-1) of X subsets and tested on 1 or more of said subsets.

The individual predictions may be a series of predictions, such as each of the series is a prediction on one biological sequence, and the weighting may comprise an assessment of the relative accuracy of substantially each individual prediction or each individual prediction means on substantially all, or one or more subsets of the predictions in a series of predictions. Preferably, this weighting of particular individual predictions means results in an assessment that the certain predictions rendered by the systems on substantially all or one or more of the subsets of the predictions in a series of predictions are to be excluded from the weighted average, and that the individual prediction means in question is/are to be excluded from the weighted average in further predictions, either with respect to substantially all or with respect to one or more of the subsets of the predictions in a series of predictions. Thus, the prediction system may comprise substantially only the prediction means not excluded by the assessment. The number of prediction means not excluded being at least 3 such as 4, preferably at least 5, 6, 7, 8, 9, or 10, particularly 10.

In preferred embodiments, the output of one level of prediction means comprises a descriptor of 2, 3, 4, 5, 6, 7, 8 or 9 consecutive residues, preferably 3, 5, 7, or 9 consecutive residues.

The assessment of the accuracy of a prediction means and or a prediction of may preferably be on the basis of combining the predictions provided by the individual prediction means on the basis of predictions provided by either substantially all or all prediction means of the system or substantially all or all prediction means of the system which do not compromise the accuracy of the combined prediction or substantially all or all prediction means of the system which are accurate above a given value or substantially all or all prediction means of the system which are estimated to be accurate above a given confidence rating.

The weighted network outputs are averaged by a per-chain, per-subset of a chain, or per-residue confidence rating. The per-residue confidence rating is typically calculated as the average per residue absolute difference between the highest probability and the second highest probability whereas the per-subset of a chain confidence rating or per-chain confidence rating is calculated by multiplying each component of a single- or multi-component output for each residue, said output produced by the selected prediction means by the per-chain estimated accuracy obtained for said chain and prediction means, and the resulting products summed by residue and component, and the resulting sums being divided by the sum of weights, and the resulting maximal per-residue component quotient being used to determine the H or E or C secondary structure assignment for that residue, and the per-chain per-prediction probability in the H versus E versus C assignment is averaged over a given protein chain.

A standard feed forward neural network may be used comprising of one hidden layer. As is known by the person skilled in the art, initial weights may be adjusted by a conventional back propagation procedure (Rummelhart, D., Hinton, G. & Williams, R. Learning internal representations by error propagation. In D. Rumelhart and J. McClelland, editors, Parallel Distributed Processing, 1:318-363. MIT Press (1986)). Details regarding the implementation of neural networks for the analysis of sequences such as biological sequences is also known by the person skilled in the art.

A particularly attractive embodiment of the method comprises a first level of neural network (termed a sequence-to-structure network) with four different window sizes (15, 17, 19, 21) and two different numbers of hidden units (50 and 75), resulting in eight different network architectures. The neural network operates on numbers when predicting an output based on input. Input must therefore be converted to one or more binary or real numbers before being fed in to the network, and the output from a network is one or more numbers, which in one particularly attractive embodiment may be interpreted as propensities for H, E, and/or C. For a protein sequence, each amino acid in the window is encoded with 20 neurons, represented as a sequence profile, and an additional twenty first neuron representing the end of a sequence. Four additional input neurons are used to represent the length L of the protein chain, and the position in the sequence P of the central amino acid in the window, given as L/1000, 1-L/1000, P/L, 1-P/L. Also, 20 input neurons are used to represent the amino acid composition of the chain. Nine output neurons are used, three for the central amino acid in the window and three for each of the amino acids flanking it. For each of these amino acids three output neurons were used representing alpha-helix, extended strand, and coil, respectively.

The neural networks are trained using a ten-fold cross-validation procedure, i.e. it is trained on nine of the ten subsets and tested on the last tenthsubset. Thus, 80 different sequence-to-structure networks are trained.

For each of the initial 8 architectures of the networks, ten structure-to-structure networks are trained, thus 80 different structure to structure networks were trained. In this embodiment, all structure-to-structure networks have a 17 residue input window and 40 hidden units. The window size and number of hidden units in this embodiment should not be construed as limiting.

A novel sequence passes first the 80 sequence-to-structure networks, then each these predictions are passed through the ten structure-to-structure networks resulting in 800 networks (and 800 predictions and outputs).

Prediction and Output

The output generated by each of the levels may be a single or multi-component prediction. A non-limiting example of a single component prediction is a value ascribed to an angle of a bond, or to a constant relating to or reflecting hydrophobicity, hydrophilicity, acidity, basicity, nucleophilicity, electrophilicity, polarity, dectron density or rotational freedom, interatomic distance, bond strength, scalar products of atomic vectors, cross products of atomic vectors, angles between atomic vectors, triple scalar products between atomic vectors, torsion angles, atomic angles such as but not exclusively omega, psi, phi, chi1, chi2, chi21, chi3, chi4, chi5 angles, chain curvature, chain torsion angles, and mathematical functions thereof.

The chemical, physical or biological features related to chemical substances or to chemical interactions to be predicted are typically descriptors of molecules or subsets of molecules.

In general, the input data and/or its features have a corresponding or complementary output data. Moreover, the input elements can be arranged in one or more sequences, such as amino acid residue or nucleotide residue in a peptide or nucleic acid, and that for each input element, predictions are made for more than one output element.

Furthermore, the more than one output elements typically correspond to neighbouring input elements.

In preferred embodiments, the output of one level of prediction means comprises a descriptor of 2, 3, 4, 5, 6, 7, 8 or 9 consecutive residues, preferably 3, 5, 7, or 9 consecutive residues.

Mutli-component prediction may be a combination of related single component predictions or relate to secondary structure, secondary structure class assignment, or tertiary structure. An example of a multi-component secondary structure class assignment comprises a per-residue, per-chain, or per-subset-of-chain prediction of the preponderance of a residue, chain, or subset-of-chain to comprise or to be comprised in a helix, a coil and an extended chain.

A multi-component prediction comprises of an least 2-component prediction, such as a 3-, 4-, 5-, 6-, 7-, 8-, 9-, or 10-component prediction. Typical 3-component predictions may comprise of a prediction for a helix (H), a coil (C), and extended strand (E).

Single- or multi-component output from at least one neural networks in at least one level in a hierarchical arrangement of levels of neural networks is preferably supplied as input to more than one neural network in a subsequent level of neural networks.

The weighting or its corresponding weighted average comprises a multiplication of each component of a single- or multi-component for each residue, said output produced by the selected prediction means by a per-sequence estimated performance obtained for said chain and prediction means, and the resulting said products summed for each residue and component, and the resulting sums being divided by the sum of weights and the resulting maximal per-residue component quotient being used to determine the descriptor said residue, and the per-sequence per-prediction probability of the descriptor is averaged over a given protein chain.

Each prediction is assigned a weight and a weighted average comprises an evaluation of the estimation of the prediction accuracy for a sequence, such as a protein chain, by a prediction means. The estimation of the prediction accuracy of a protein sequence may be made by summing the per-residue maximum of H versus E versus C probabilities for said protein chain and dividing by the number of amino-acid residues in the protein chain and the mean and standard deviation of the accuracy estimation may be taken for all prediction means for the protein chain, and a weighted average may be made for substantially all or optionally a subset of prediction means, wherein the subset comprises those prediction means with estimated accuracy above a threshold consisting of the mean estimated accuracy, the mean accuracy plus one standard deviation above the mean accuracy, or the mean estimated accuracy plus two standard deviations above the mean, or wherein the subset comprises at least N prediction means, such as 10, in cases where the accuracy of fewer than 10 estimated predictions fail to satisfy the threshold.

The output of each of the neural networks undergo conversion into probabilities. The outputs from each prediction for each network are normalised so they sum one.

A prediction for each sequence in the TT set may be made using the 800 combinations of networks. A histogram may be made for each of the 800 combinations so that the neural network outputs could be converted into probabilities. The conversion into probabilities for one combination is done by first normalising the outputs by dividing each of the outputs (H, E, and C) by the sum of the three outputs. The range of values that each output can be in after normalisation is between zero and one. This range is divided into 20, such that a combination of outputs for H and E falls within one of 20*20=400 bins. For each of these bins the probability for H, E, and C is calculated by calculating the number of times that the correct output is H, E, or C, respectively, divided with the number of times that the predicted output for H and E falls within this bin. Other methods of converting output into probabilities are easily anticipated, such as using the soft-max energy function in neural networks, especially by the person skilled in the art.

A balloting of neural network outputs is made in order to make a prediction on a query sequence. A query sequence may be run through the 800 network combinations as described above. In the embodiment where a per-residue confidence rating of an output was made, the confidence of each network on the query sequence is calculated as the average per-residue absolute difference between the largest and the second largest probability. Typically, only networks having a confidence of at least one standard deviation above the mean, such as two, may be used in the balloting. However, the ten most confident networks are typically used. The probability of a given secondary structure class may be calculated as the per-chain confidence weighted average probability for that class over the networks participating in the balloting. The residues are assigned to be in the secondary structure class having the largest predicted probability.

In order to measure the prediction accuracy, it may be calculated as the so-called Q3 performance. The Q3 performance is calculated as an average accuracy over the chains in the test set. For each said chain, the accuracy is calculated as (the number of residues which are predicted to be in the correct class divided by the number of residues in the protein) times 100%. The evaluation set may be the RS set.

Common for all the different aspect of the present invention is that the invention may further comprise predicting a set of features of input data where the input data provided to a first level of neural networks is further inputted to the subsequent levels of neural networks.

Furthermore, a prediction system may advantageously be established by implementing the methods according to the various aspect of the invention in a computer system comprising storage means, such as memory, hard disk or the like and computation means, such as one or more processor units. Furthermore, a prediction system established by a system comprising storage means, such as memory, hard disk or the like and computation means, such as one or more processor units being able to perform the difference steps according to the present invention is preferred and advantageous. A prediction system comprising a combination of systems established by the methods according to the present invention or comprising a combination of systems established by the method according to the present invention and another type of system is preferred and advantageous.

In the following the present invention and in particular preferred embodiments thereof are further described with reference to the figures and tables.

### BRIEF DESCRIPTION OF THE DRAWINGS AND THE TABLES

Table 1: Example of generation of input and output examples.

For each amino acid in each sequence a prediction is made. During training the correct output is furthermore used to adjust the weights in the neural network. In order to do this a corresponding input-output example must be made for each amino acid in each sequence.

In this example, the sequence: GYFCESCRKI

and the corresponding secondary structure: . . . HHHHHHHH

is used. An input window of 3 amino acids have been used. This means that when the secondary structure for the N'th amino acid in the sequence is to be predicted, the N-1th, the Ntn and the N+1th amino acid is given to the neural network as input. No output expansion have been applied, meaning that it is only the secondary structure for the central amino acid in the input window (the Nth) which is predicted. In this example, the input sequence is ten amino acids long and there are therefore ten corresponding input output examples. These four of these examples are shown in the table. The conversion from amino acids and secondary structure classes to numbers are illustrated in table 3 and 4, respectively.

TABLE 2
Generation of input and output examples using the
same sequence and secondary structure

| | Input | output |
|---|---|---|
| Example 1 | -GY | . |
| Example 2 | GYF | . |
| Example 3 | YFC | H |
| . . . | | |
| Example 10 | KI- | H |

As in Table 1, an input window of 3 amino acids have been used. Output expansion have been applied, using an output window of three. This means that when the central amino acid in the input window is the Nth amino acid, a prediction of the secondary structure is not only made for the Nth amino acid but a prediction is also made for the N-1th amino acid and for the N+1th amino acid.

TABLE 3
Conversion from amino acids to binary descriptors.

| | Input | output |
|---|---|---|
| Example 1 | -GY | -.H |
| Example 2 | GYF | ..H |
| Example 3 | YFC | .HH |
| . . . | | |
| Example 10 | KI- | HH- |

Table 3: Conversion from amino acids to binary descriptors.

Each amino acid in the input window is converted into 21 numbers, each of which are fed into one unit in the input layer of the neural network. The 21^{th} number is set to one if the position in the window is outside the sequence (represented in the table as the amino acid “-”) and zero otherwise. The 20 first numbers represent the amino acid. The 20 numbers might also be real numbers rather than integers. They may thus represent the frequency of an amino acid in a position in a multiple alignment or mathematical functions hereof, such as the log-odds ratio of the probability of finding a particular amino acid in that position in an multiple alignment.

TABLE 4
Conversion from secondary structure to number descriptors.

| Amino acid | Number representation |
|---|---|
| A | 100000000000000000000 |
| C | 010000000000000000000 |
| . . . | |
| - | 000000000000000000001 |

Table 4: Conversion from secondary structure to number descriptors.

In this example zeros and ones is used, but the secondary structure may in general be represents by real numbers rather than binary numbers.

| Secondary structure | Binary representation |
|---|---|
| H | 100 |
| E | 010 |
| C | 001 |

FIG. 1: Schematic drawing of the information flow.

The input is fed into the prediction system which produces an output.

FIG. 2: Schematic drawing of a prediction system.

The input is fed into each of the level 1 predictors. Different subtypes of the input may be fed into the different level 1 predictors. The output of each of these predictors is in turn fed as input into one ore more level 2 predictors. The level 2 predictors may also take subtypes of the input fed or not fed into the level 1 predictors as additional input. The output from the level 2 predictors is then combined to produce the final output.

FIG. 3: Schematic drawing of a neural network.

The input amino acid sequence is YACES. In this example the neural network has a input window which spans three amino acids. In the example the three letters A, C, and E is fed into the neural network. Please note that each amino acid is represented to the neural networks as 21 numbers as described in table 3, and that each of the three boxes show in the input layers thus represents 21 input units. The neural network depicted has two hidden units and three output units. The three output units shown in this example represents Helix (H), Extended strand (E) and Coil (C).

FIG. 4: Schematic drawing of the input to the second level networks.

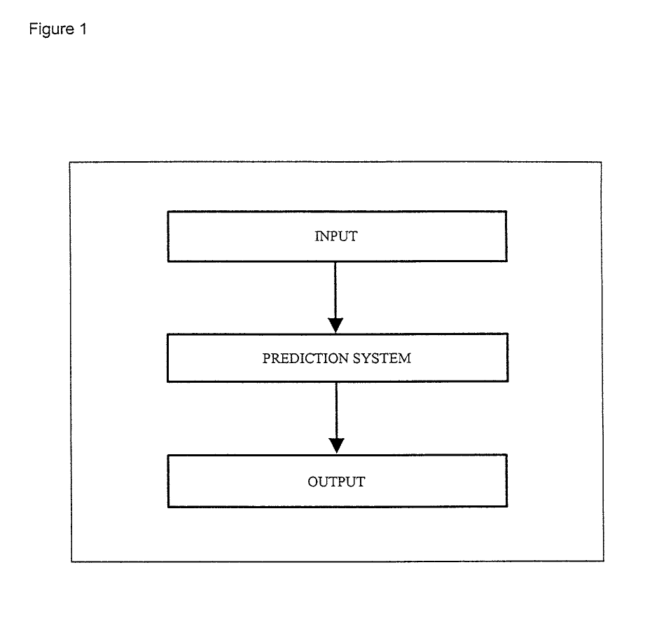

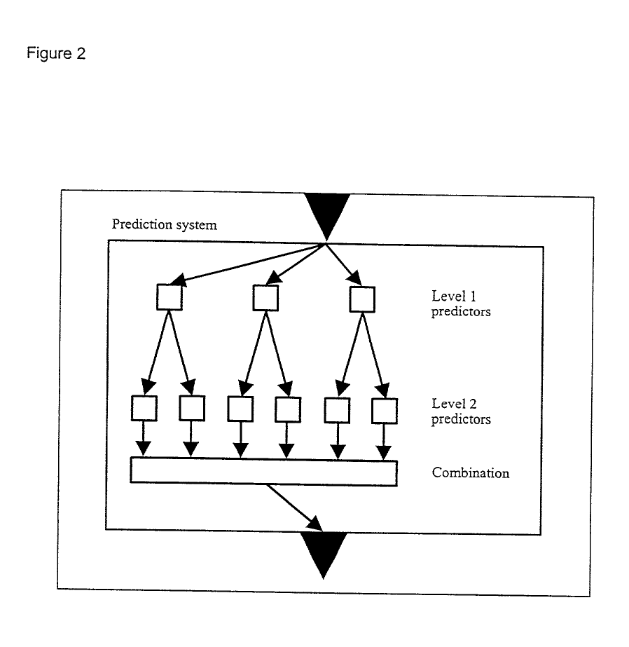

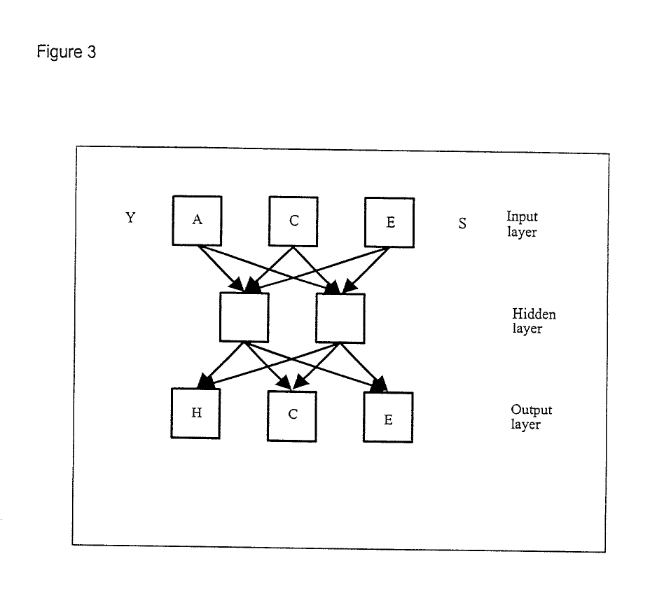

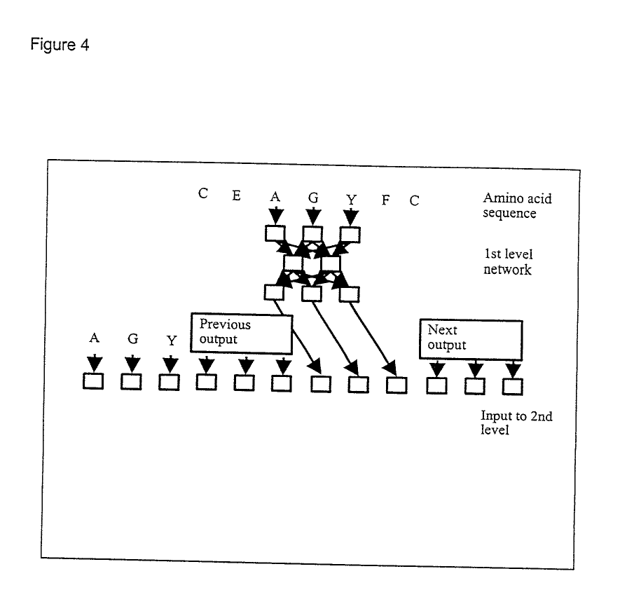

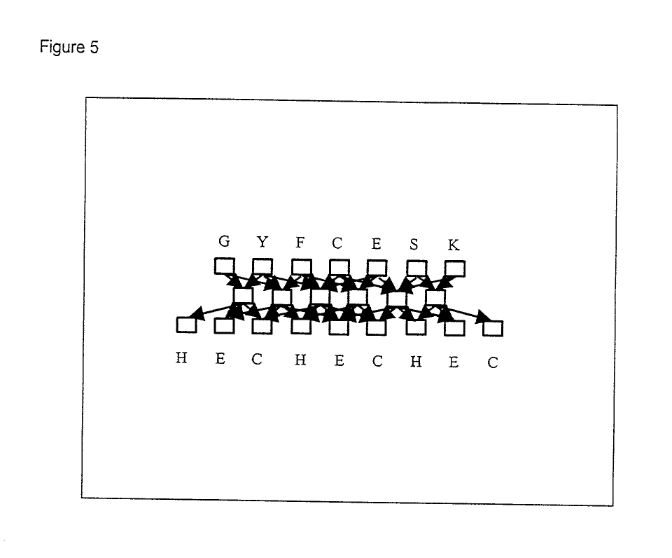

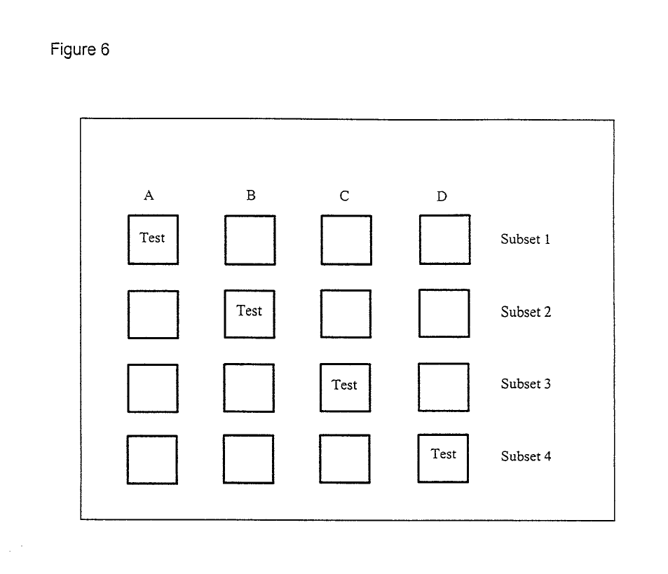

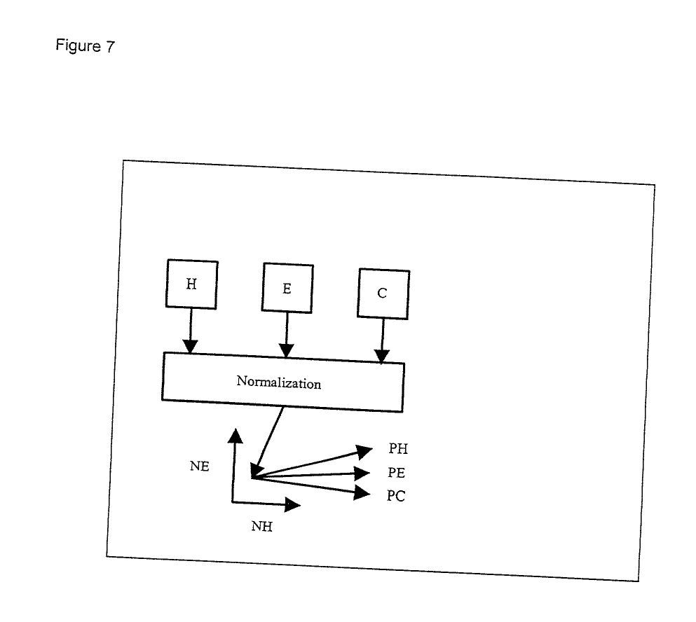

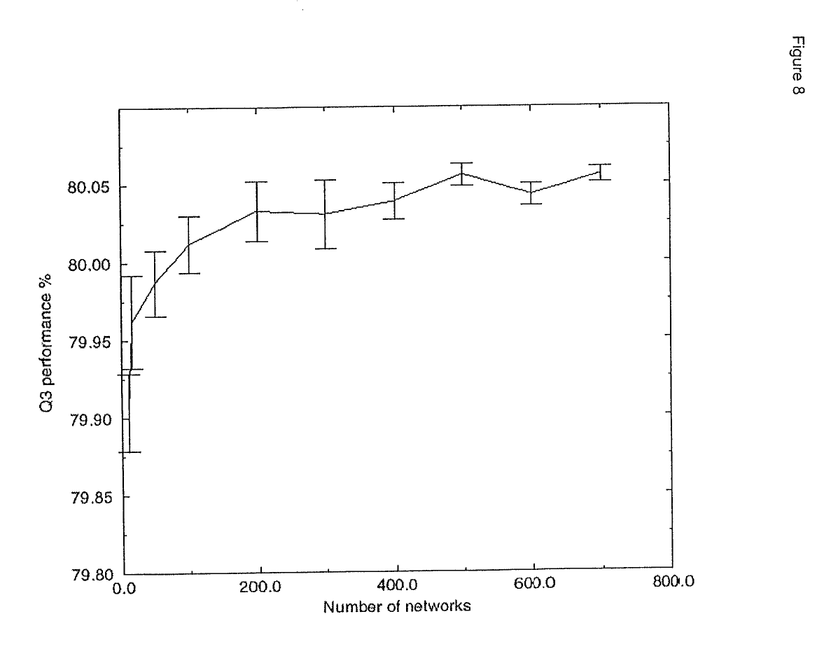

The amino acid sequence “CEAGYFC” is fed into the 1^{st} level network. In this example the 1^{st} level network has an input window of three amino acids. For each triplet of amino acids {-CE, CEA, EAG, . . . FC-} the 1^{st} level network produces three outputs e.g. For H, E and C. The figure depicts how the input to the second level network is prepared in order for it to make a prediction for G in the amino acid sequence. The second level network not only takes the output from the first level network with “AGY” fed into input window, but also previous output from the first level network (with “EAG” in the input window), and the next output from the first level network (with “GYF” in the input window). In general the second level network may take N previous predictions and M next predictions as input and thus have an input window of N+M+1 outputs from the first level networks. In the example the second level network takes an additional input of three amino acids. In general it may take an input of any number of amino acids. The amino acids can be represented to the network as described in table 3. On both levels the neural networks may take a number of additional inputs, which can for example represent the length of the sequence, or the amino acid composition of the sequence.

FIG. 5: Schematic drawing of a neural network with output expansion.

The neural network in the example gets the amino acid sequence GYFCESK as input. In this example the network predicts the secondary structure for three consecutive residues in the input sequence. The leftmost “HEC” represents the predicted secondary structure for “F” in the input sequence, the middle “HEC” represents the predicted secondary structure for “C” in the input sequence, and the rightmost “HEC” represents the predicted secondary structure for “E” in the input sequence. Output expansion may in general represent the predictions for any number of amino acids in the input sequence, and thus not only represent the output descriptors related to three amino acids as in this example.

FIG. 6: Schematic depiction of the cross validation procedure.

The figure depicts a four fold cross validation procedure. The data set is divided into four subsets. In each of the four crossvalidations (A, B, C, and D) a different subset is selected as the test set and the methods are trained on the three remaining subsets. The crossvalidated performance is the average performance on the subsets used as test sets.

FIG. 7: Schematic drawing of the post processing of the output from the neural networks.

First each of the N outputs (in this case three: H, E, and C) may be divided by the sum of the N outputs, in order to normalise them. Thereafter the normalised outputs (NH, NE, and NC) is converted into probabilities (PH, PE, and PC). This conversion may be done by empirically determining the mathematical relation between the normalised output and the probabilities.

FIG. 8: The Q3 score as a function of the number (N) of neural network predictions included in the balloting procedure.

For each data point on the graph the average and standard error of ten random selections with replacement is shown.

In the following, the present invention will be described in greater details and in particular preferred embodiments thereof in connection with the accompanying figures.

### DETAILED DESCRIPTION OF PREFERRED EMBODIMENTS OF THE PRESENT INVENTION

The structure prediction system developed, by use of novel methods such as output expansion and a balloting procedure results in an overall Q3 performance in secondary structure prediction of 80.1%, when evaluated on a commonly used test set of 126 protein chains.

A new method called output expansion allows for increases in prediction system performances in general.

A new balloting procedure efficiently combines information from 800 neural network predictions.

The 800 predictions preferably arise from a 10 fold cross-validated training and testing of protein sequences on a primary neural network and a second filtering neural network.

Eighth different neural network architectures are preferably used in the secondary structure prediction system.

The prediction of secondary structure is preferably performed on three consecutive residues at a time.

The use a neural network algorithm for secondary structure prediction is preferred given this has led to an increase Q3 performance (Rost & Sander, 1993; Jones, D. T. Protein secondary structure prediction based on position-specific scoring matrices. J. Mol. Biol., 292: 195-202 (1999)).

The assessment of an increased performance is based on the commonly used evaluation set of 126 protein chains, the RS126 set (Rost & Sander, 1993). For each of the prediction systems, the Q3 performance may be measured using this set as a test set. Neural networks are trained with a 10 fold cross-validation procedure and only on a set of protein chains that are non-homologous to the RS126 set. This training set contains 1032 protein chains.

The combination of 800 network predictions using the balloting scheme lead to a Q3 score of 80.1%. The percentage of correct predictions were 84.6%, 69.0%, and 82.2% with correlation coefficients of 0.778, 0.639, and 0.623 for H, E and C, respectively. The effect of using different numbers of networks in the balloting procedure is shown in FIG. 8. The performance is seen to continue to increase as more networks are included in the balloting process.

In FIG. 8, the Q3 score as a function of the number (N) of neural network predictions included in the balloting procedure. For each data point on the graph the average and standard error of ten random selections with replacement is shown.

Two similar neural network trainings may be performed with and without the use of output expansion. It is difficult to improve on an already good neural network performance but the use of output expansion followed by a straight averaging of 800 predictions lead to a Q3 score of 79.9% with output expansion as compared to 79.7% without output expansion.

An increase in the accuracy of secondary structure prediction may be obtained by combining many neural network predictions.

Critically, an increase in the Q3 score may be obtained using a novel procedure called output expansion i.e. prediction of the secondary structure for more than one consecutive residue at the time. These additional output neurons give hints to the neural networks by restraining the weights in the neural networks.

Preparation of Data Sets

Data used to train the neural networks may be prepared from atomic coordinate files available in the Protein Data Bank (Aug. 1999) (Bernstein, F. C. and Koetzle, T. F., Williams, G. J. B., Meyer Jr., E. F., Brice, M. D., Rodgers, J. R., Kennard, O., Shimanouchi, T., Tasumi, M. The protein data bank: A computer based Archival file for macromolecular structures. J. Mol. Biol. 112:535-542 (1977)). The files with database entries may, at the time of filing, be downloaded to a local computer by ftp from the website http://www.rcsb.org/pdb/cgi/ftpd.cgi. The criteria applied to include protein chains in the data set are i) a resolution better than or equal 2.5 Å for crystal structures and for NMR structures only regions where models superimpose with a root mean square deviation less than or equal to 1 Å. The remaining subset of protein chains are included, provided a chain length longer than 29, no occurrence of chain breaks as defined in the DSSP program (Kabsch & Sander, 1983). These criteria results in a set of 9926 protein chains which is homology reduced by use of the Hobohm algorithm #1 (Hobohm et at 1992) to a set of 1168 chains. The homology reduction is performed by first sorting the chains according to their resolution, thereby producing a list where chains with the best (lowest) resolution comes first. A homology reduced set is hereafter constructed using an iterative procedure with two steps: 1. The first on the list is moved to the homology reduced set; 2. All sequences with a similarity above a threshold to the first on the list are thereafter removed from the list. Steps 1 and 2 are repeated until no chains are left on the list.

The similarity between two chains is determined by first aligning the sequences of the two chains against each other using the program ssearch where the penalty for opening a gap is set to −12, and for extending a gap is set to −4. The pam120 scoring matrix is used to measure the similarities between different amino acids. This matrix may be found in the file pam120.mat from the fasta package. The fasta package can be downloaded from the website: “ftp://ftp.bio.indiana.edu/molbio/search/fastaf”. The similarity may be calculated by running the ssearch program from the fasta package with the command line “ssearch-s pam120.mat-f -12-g -4 chain1.fasta chain2.fasta”, where chain1.fasta and chain2.fasta is the names of two files containing the sequence of the chains in fasta format, respectively. A file in fasta format may contain one or more entries. Each entry has a header line containing a “>” character followed by a name of the entry, and optionally a description. This header line is then followed by the amino acid sequence in a one-character-per-amino-acid code, with 60 amino acids per line. The threshold for similarity is defined by that the percentage of sequence identity in the alignment (I) must be above 290/sqrt(L), where L is the length of the alignment. Finally, transmembrane proteins are removed and chains with homology above our threshold to sequences in the RS126 set, giving a set of 1032 protein chains, to be used for training of all subsequent neural networks.

An unbiased measure of performance secondary structure predictions relies on the selection of the sequence similarity. The sequence similarity reduction preferably relies on a pairwise sequence alignment where sequence identity must be below 290/L, where L is the alignment length. This threshold closely resembles the threshold developed by Sander and Schneider (1991), i.e. that local alignments above the threshold usually have a three state secondary structure identity above 70%, and an RMS below 2.5 Å. The degree of homology allowed is thus comparable to that in the set used by Rost & Sander, 1993 (the RS126 set), and enables comparison of the results obtained with the ones obtained by using the RS126 set (Rost & Sander, 1993).

Sequence Profiles

Sequence profiles are typically generated with the program PSI-BLAST package version 2.0.3 (Altschul,1991, ftp://ncbi.nlm.nih.gov/blast/). The program may be run using the command line “blastpgp -i sequence.fasta -d Blastdatabase -b 0 j 3”, where sequence.fasta is the name of the query sequence in fasta format and Blastdatabase is the name of the blast database. The blast database may be generated from a non-redundant database comprised of sequences from Swissprot and Trembi (Bairoch & Apweiler, 1996). This database is pre-processed such that residues in the protein sequences annotated as RICH, COIL, REPEAT, HYDROPHOBIC, SIGNAL, or TRANSMEMBRANE, were substituted with an X, to avoid picking up to many low information sequences with blastpgp. These sequences is then first converted into fasta format, and then converted to the blast database format using the formatdb program from the PSI-BLAST package version 2.0.3 (Altschul, 1991). This may be done by issuing the command the command “formatdb -i fasta_file”. Profiles are extracted from the output from the blastpgp program and saved in a file. The last log-odds matrix produced by the program is used as the profile for the sequence. If no such matrix is produced by the program, the profile may be made from a blosum62 matrix (Henikoff and Henikoff, 1992). This may be done by for each amino acid in the sequence to extract the row in the blosum62 matrix corresponding to this amino acid.

DSSP Assignment and Output Expansion

The neural networks are trained against a reduced sets of DSSP assignments. The eight DSSP categories are reassigned into three states being, pure helix H, strand E and all remaining categories assigned to coil C. Neural networks are trained on three output categories H, E and C, when the output expansion mode is turned off. Training with output expansion results in nine output categories as the assignment of the central residue i in a window, becomes dependent on the three-state assignment of its neighbour residues at positions i−1 and i+1, respectively. An example of the output expansion assignment scheme is shown in Table 1.

TABLE 1
Assignment scheme for a protein sequence with
and without output expansion.

| | Assignment without output | Assignment with output |
|---|---|---|
| Primary sequence | expansion | expansion |
| 1A | C | -CC |
| 2G | C | CCH |
| 3W | H | CHH |
| 4A | H | HHC |
| 5L | C | HCE |
| 6I | E | CE- |

Neural Networks

A standard feed forward neural network may used with one hidden layer and/or weights updated by a conventional back propagation procedure (Rummelhart, 1986). In the first level of neural networks, the so called sequence to structure networks architectures with window sizes of 15, 17, 19 and 21 in combination with 50 and 75 hidden units were used. The amino acids may be encoded from the sequence profiles into 20 neurons as the log-odd ratios and a 21st neuron represents end of sequence. In addition two neurons are used to store the relative position in the protein sequence i/L and 1−i/L, where L is the length of the protein chain and i is the position of the central residue in the window. Also, the relative size of the protein is encoded as S/Max and 1−S/Max, where S is the length of the protein and Max represents the longest protein chain in the database. Finally 20 additional neurons may be encoded as the fraction of the 20 amino acids for a given protein. The output layer comprises nine neurons due to training with output expansion. Output from the primary neural network may be passed into a second neural network with a window size of 17 and 40 hidden units.

The primary neural networks are trained using a ten fold cross validation procedure, i.e. training on nine tenth and testing on one tenth. As training is performed on eight different architectures, each ten fold cross validated, a total of 80 primary networks are obtained. For each architecture, the ten tenth of output activities are reassembled and used as input to a second neural network. Again each of the eight new sets is passed to the second neural network and training is performed with a cross validation procedure similar to that of the primary networks. The input to the second neural network, the structure to structure network, are 20 neurons encoded with the binary amino acid representation, a 21st neuron representing end of sequence and 9 neurons represented by the output activities from the primary neural network. Training of the structure to structure networks also produce 80 trained networks.

Secondary structure predictions on a protein sequence first pass through each of the 80 primary networks giving 80 predictions. Each of these 80 predictions are hereafter passed to the correct 10 structure to structure networks, giving a total of 800 secondary structure predictions. Probability matrixes are made for each of the 800 predictions, such that output activities is transformed into a probability. These matrices are only made once after training all the networks. Hereafter output activities produced for a query sequence are transformed via the matrices into probabilities.

Balloting Probabilities

The balloting procedure is a statistical method that enables an efficient combination of multiple predictions. The procedure consists of two steps. First, per residue confidence aᵢⱼₖ is associated with each residue i in chain j for prediction k, as the highest minus the second highest of the three probabilities Pᵢⱼₖ(H), Pᵢⱼₖ(E) and Pᵢⱼₖ(C). A mean confidence for prediction k on chain j is calculated:

αⱼₖ=1/NⱼΣαᵢⱼₖ

where the sum is over all residues i=1 . . . Nⱼ in chain j. Furthermore a mean and standard deviation for per chain confidence is calculated:

<αⱼ>=1/NₖΣαⱼₖ

σⱼ=√(<αⱼ²>−<αⱼ>²)

where the sum is over all predictions k. The probability Pᵢⱼ(class) for residue i in chain jis calculated:

Pᵢⱼ(class)=ΣαⱼₖPᵢⱼₖ(class)/Σαⱼₖ

where class is H, E or C, and the sum is over a subset of prediction sets k for which αⱼₖ is greater than <αⱼ>+σⱼ, but with the constraint that at least 10 prediction sets k are included in the weighted average.

Distance Class Prediction

A neural network by the present invention is able to predict distances between C alpha atoms and may use the output from such networks as input to a secondary structure prediction network. The preliminary result is that this increases the performance of the secondary structure prediction by approximately one percentage point.

The procedure is presented in the following:

Prediction of distance classes has been performed for a sequence separation of 4. The three distance classes A, B and C are defined as:

A: d<6.66AA

B: 6.66<=d<11.01 AA

C d>=11.01

where d is the distance between CA atoms CA(i)−>CA(i+4).

The window is non-overlappig and spanning 13 residues from residue 1−4 to 1+8. The sequence profile is used as input and three probabilities describing P(H), P(E) and P(C). Additional information from the amino acid composition, the relative amino acid position and the relative size of the protein is used as input to the neural network. The number of hidden units is 50.

For secondary structure prediction, a 10-fold cross-validation training is performed on pef8.2.nrs, using a window size of 15 and 50 hidden units. The input is the sequence profile and three activities obtained from the distance class prediction. The amino acid composition, relative amino acid position, relative protein size are also used as input the neural network. The training is performed using output expansion with one residue at each side.

### EXAMPLE OF A PRACTICAL IMPLEMENTATION OF THE INVENTION

In preferred embodiments, the present invention has been implemented as a computer program that is executed on a computer. The programming languages per, C, fortran and shell script have been used to implement the invention. The program can be executed on an Octane or an O2 computer from silicon graphics, with 8 gigabyte hard disk, and 384 megabyte RAM, running the IRIX 6.5 operating system. The program have also been installed on a computer with a 266 Mhz pentium II processor from intel with 8 gigabyte hard disk, and 512 megabytes RAM, running the RedHat 6.2 version of the Linux operating system. The program has been implemented in such a way that part of the calculations may be run in parallel on two or more processors.

The program may with minor modifications run on other types of computers such as computers from different manufactures or computers with different hardware configurations, or on computers running different operating systems or on two or more different computers. The program may also be implemented using other programming languages.

1. A method for predicting a set of chemical, physical or biological features related to chemical substances or related to interactions of chemical substances
using a system comprising a plurality of prediction means, the method comprising
using at least 16 different individual prediction means, thereby providing an individual prediction of the set of features for each of the individual prediction means and
predicting the set of features on the basis of combining the individual predictions,
the combining being performed in such a manner that the combined prediction is more accurate on a test set than substantially any of the predictions of the individual prediction means.

2. A method according to claim 1, wherein the combining being performed is an averaging and/or weighted averaging process.

3. A method according to claim 1, wherein the combining of the predictions provided by the individual prediction means are based on predictions provided by either
substantially all or all prediction means of the system or
substantially all or all prediction means of the system which do not compromise the accuracy of the combined prediction or
substantially all or all prediction means of the system which are accurate above a given value or
substantially all or all prediction means of the system which are estimated to be accurate above a given confidence rating.

4. A method according to claim 1, wherein the number of different predictions means is at least 20, such as at least 30, such as at least 40, 50, 75, 100, 200, 400, 600, 800, 1000, 1200, 1400, 1600, 1800, 2000, 2500, 3000, 4000, 5000, 6000, 7000, 8000, 9000, 10,000, 15,000, 20,000, 30,000, 40,000, 50,000, 100,000, 200,000, 500,000, 1,000,000.

5. A method according to claim 1, wherein the type of prediction means are selected from the group consisting of neural networks, hidden Markov models (HMM), EM algorithms, weight matrices, decision trees, fuzzy logic, dynamical programming, nearest neighbour approaches, and vector support machines.

6. A method according to claim 1, wherein the prediction means are diverse with respect to type, and/or with respect to architecture, and/or in case of prediction means subjected to training with respect to initial conditions, and/or with respect to training.

7. A method according to claim 2, wherein the weighted averaging process is performed based on the accuracy of substantially each or each of the individual prediction means.

8. A method according to claim 7, wherein the individual predictions performed are a series of predictions, and the weighting comprises an evaluation of the relative accuracy of substantially each individual prediction or each individual prediction means on substantially all, or one or more subsets of the predictions in a series of predictions.

9. A method according to claim 8, wherein the weighting of particular individual predictions means results in an evaluation the predictions rendered by the systemson substantially all or one or more of the subsets of the predictions in a series of predictions are to be excluded from the weighted average, and the individual prediction means in question is/are excluded from the weighted average in further predictions, either with respect to substantially all or with respect to one or more of the subsets of the predictions in a series of predictions.

10. A method according to claim 3, wherein the confidence rating is calculated by multiplying each component of an individual prediction of the selected prediction means
by the weight obtained for a sequence and prediction means,
the resulting product summed for each component of each residue over all prediction means,
the resulting sums being divided by the sum of weights, and
the resulting maximal per-residue component quotient being used to determine the H or E or C secondary structure assignment for that residue.

11. A method according to claim 9, wherein the number of prediction means not excluded being at least 3 such as 4, preferably at least 5, 6, 7, 8, 9, or 10.

12. A method according to claim 10, wherein the number of prediction means not excluded being at least 3 such as 4, preferably at least 5, 6, 7, 8, 9, or 10.

13. A method for establishing a prediction system for predicting a set of chemical, physical or biological features related to chemical substances or to chemical interactions represented by an input data using a system comprising a plurality of prediction means, the method comprises performing the steps according to claim 1.

14. A method according to claim 1, wherein the prediction means comprise neural networks.

15. A method according to claim 14, wherein the neural networks are different with respect to architecture, and/or with respect to initial conditions, and/or with respect to selection of training set, and/or with respect to learning rate and/or with respect to subtypes of input data fed to respective neural networks, and/or with respect to subtypes of output data sets rendered by the respective neural networks.

16. A method according to claim 1, wherein the chemical, physical or biological features related to chemical substances or to chemical interactions to be predicted are descriptors of molecules or subsets of molecules.

17. A method according to claim 16, wherein descriptors are selected from the group comprising secondary structure class assignment, tertiary structure, interatomic distance, bond strength, bond angle, descriptors relating to or reflecting hydrophobicity, hydrophilicity, acidity, basicity, relative nucleophilicity, relative electrophilicity, electron density or rotational freedom, scalar products of atomic vectors, cross products of atomic vectors, angles between atomic vectors, triple scalar products between atomic vectors, torsion angles, atomic angles such as but not exclusively omega, psi, phi, chi1, chi2, chi3, chi4, chi5 angles, chain curvature, chain torsion angles, and mathematical functions thereof.

18. A method according claim 16, wherein molecules are selected from the group comprising proteins, polypeptides, oligopeptides, protein analogues, peptidomimietic, peptide isosteres, pseudopeptide, nucleotides and derivatives thereof, PNA and nucleic acids.

19. A method according claim 18, wherein molecules are selected from the group comprising proteins, peptides, polypeptides and oligopeptides.

20. A method according to claim 1, wherein the prediction means of the system are arranged in levels and wherein at least one subtype of data provided by a first level of prediction means is transferred changed or unchanged to at least one subsequent level.

21. A method according to claim 20, wherein the at least one subtype of data transferred to the at least one subsequent level comprises subsets of predictions provded by the first level of prediction means and/or subtypes of input data either changed or unchanged from input data fed into the first neural network system.

22. A method according to claim 20, wherein subtypes of input data are selected from the group comprising amino acid sequence, nucleic acid sequence, sequence profile, amino acid composition, nucleic acid composition, window, window size, length of protein, length of nucleotide, and descriptor.

23. A method according to claim 13, wherein input data comprises input elements each having a corresponding output element, and the input elements may be arranged in one or more sequences, such as an amino acid residue or a nucleotide residue in a peptide or nucleic acid sequence, and that for each input element, predictions are made for more than one output element.

24. A method according to claim 23, wherein the more than one output elements correspond to neighbouring input elements.

25. A method for prediction of descriptors of protein structures or substructures comprising
feeding input data representing at least one residue of a protein sequence to at least 16 diverse neural networks arranged in parallel in a first level,
generating by use of the networks arranged in the first level a single- or a multi-component output for each networks the single- or multi-component output representing a descriptor of one residue comprised in the protein sequence represented in the input data, or the single- or multi-component output representing a descriptor of 2 or more consecutive residues of the protein sequence,
providing the single- or multi-component output from each network of the first level as input to one or more neural networks arranged in parallel to a subsequent level(s) in a hierarchical arrangement of levels, optionally inputting one or more subsets of the protein sequence and/or substantially all of the protein sequence to the second or subsequent level(s),
generating by use of the networks arranged in the subsequent level(s) single or multi-component output data representing a descriptor for each residue in the input sequence,
weighting the output data of each neural network of the subsequent level(s) to generate a weighted average for each component of the descriptor,
optionally selecting from the multi-component output data, if generated, the component of descriptor with the highest weighted average as the predicted descriptor for each amino acid in the protein sequence, or optionally assigning a descriptor to a single-component output, and
optionally assigning the descriptor of said protein sequence.

26. A method according to claim 25, wherein the number of neural networks in one level is at least 20, such as at least 30, such as at least 40, 50, 60, 70, 80, 90, 100, 150, 200, 250, 300, 350, 400, 450, 500, 1000, 10000, 100 000 and 1 000 000.

27. A method according to claim 25, wherein the said neural networks are trained by a training process comprising an X-fold cross-validation procedure wherein each network was trained on (X−1) of X subsets of data and tested on 1 or more of said subsets.

28. A method according to claim 25, wherein the neural networks are trained by a training process comprising an 10-fold cross-validation procedure wherein each network was trained 9 of said subsets of data and tested on 1 of said subsets.

29. A method according to claim 25, wherein the neural networks are trained by a training process comprising
supplying input data, filtered or unfiltered from a database,
generating by use of the networks arranged in the first level a single- or a multi-component output for each networks, the single- or multi-component output represents a descriptor of one residue comprised in the protein sequence represented in the input data, or the single- or multi-component output represents a descriptor of 2 or more, consecutive residues of a protein sequence,
providing the single- or multi-component output from each network of the first level as input to one or more neural networks arranged in parallel in a subsequent level(s) in a hierarchical arrangement of levels, optionally inputting one or more subsets of the protein sequence and/or substantially all of the protein sequence to the subsequent level(s),
generating by use of the networks arranged in the second or subsequent level(s) a single or multi-component output representing a descriptor for each residue in the input sequence,
weighting the output of each neural network of the subsequent level(s) to generate a weighted average for each component of the descriptor, and
performing an X-fold cross-validation procedure wherein each network was trained on (X−1) of X subsets of data and tested on 1 or more subsets of data

30. A method according to claim 27, wherein X is from 2 to 1 000 0000, such as from 2 to 100 000, 2 to 10 000, 2 to 1000, 2 to 100, 2 to 50, preferably 5 to 50, such as 5, 10, 15, 20, 25, 30, 35, 40, 45 or 50.

31. A method according to claim 27 wherein the testing on the subset comprises making a prediction for each element in the data set and evaluating the accuracy of the prediction.

32. A method according to claim 25, wherein the one or more neural networks arranged in parallel to a subsequent level(s) in a hierarchical arrangement of levels comprises networks with at least two different window sizes, such at least 3, 4, 5, or 6 window sizes.

33. A method according to claim 25, wherein the one or more neural networks arranged in parallel to a subsequent level(s) in a hierarchical arrangement of levels comprises networks with at least 1 hidden unit, such as at least 2, 5, 10, 20, 30, 40, 50, 60, 75 or 100 hidden units.

34. A method according to claim 25, wherein the one or more neural networks arranged in parallel to a subsequent level(s) in a hierarchical arrangement of levels comprises networks with at least 7, such as at least 9, such as at least 11, particularly at least an 101 residue input window, such as at least 13, 15, 17, 21, 31, 41, 51, or 101 residue input window.

35. A method according to claim 25, wherein the single- or multi-component output from at least one neural networks in at least one level in a hierarchical arrangement of levels of neural networks is supplied as input to more than one neural network in a subsequent level of neural networks.

36. A method according to claim 25, wherein diverse networks are diverse with respect to architecture and/or initial conditions and/or selection of learning set, and/or position-specific learning rate, and/or subtypes of input data presented to respective neural networks, and or with respect to subtypes of output data sets rendered by the respective neural networks.

37. A method according to claim 36, wherein the networks diverse in architecture have differing window size and/or number of hidden units and/or number of output neurons.

38. A method according to claim 36, wherein the initial conditions are selected by the process of randomly setting each weight to ±0.1 and/or randomly selected from [−1; 1].

39. A method according to claim 36, wherein the learning set comprises sets generated from the X-fold cross-validation process.

40. A method according to claim 36, wherein the sub-types of input data are selected from the group comprising sequence profiles, amino acid composition, amino acid position and peptide length.

41. A method according to claim 36, wherein the sub-types of output data sets are selected from the group comprising secondary structure class assignment, tertiary structure, interatomic distance, bond strength, bond angle, descriptors relating to or reflecting hydrophobicity, hydrophilicity, acidity, basicity, relative nucleophilicity, relative electrophilicity, electron density or rotational freedom, scalar products of atomic vectors, cross products of atomic vectors, angles between atomic vectors, triple scalar products between atomic vectors, torsion angles, atomic angles such as but not exclusively omega, psi, phi, chi1, chi2, chi21, chi3, chi4, chi5 angles, chain curvature, chain torsion angles, and mathematical functions thereof.

42. A method according to claim 25, wherein the input data is taken unchanged or upon filtration through one or more quality filters from a biological database, such as a protein database, a DNA data base and an RNA database.

43. A method according to claim 25, wherein the weighted networks outputs are averaged by a per-chain, per-subset of a chain, or per-residue confidence rating.

44. A method according to claim 43, wherein the per-residue confidence rating is calculated as the average per residue absolute difference between the highest probability and the second highest probability.

45. A method according to claim 43, wherein the per-subset of a chain confidence rating or per-chain confidence rating is calculated
by multiplying each component of a single- or multi-component output for each residue, said output produced by the selected prediction means
by the per-chain estimated accuracy obtained for said chain and prediction means,
and the resulting products summed by residue and component,
and the resulting sums being divided by the sum of weights, and the resulting maximal per-residue component quotient being used to determine the H or E or C secondary structure assignment for that residue, and
the per-chain per-prediction probability in the H versus E versus C assignment is averaged over a given protein chain.

46. A method according to claim 25, wherein the output is a set number.

47. A method according to claim 25, wherein descriptors are selected from the group comprising secondary structure class assignment, tertiary structure, interatomic distance, bond strength, bond angle, descriptors relating to or reflecting hydrophobicity, hydrophilicity, acidity, basicity, relative nucleophilicity, relative electrophilicity, electron density or rotational freedom, scalar products of atomic vectors, cross products of atomic vectors, angles between atomic vectors, triple scalar products between atomic vectors, torsion angles, atomic angles such as but not exclusively omega, psi, phi, chi1, chi2, chi21, chi3, chi4, chi5 angles, chain curvature, chain torsion angles, torsion vectors and mathematical functions thereof.

48. A method according to claim 25, wherein a multi-component output comprises prediction with at least 2 components such as a 2-component, a 3-component, 4-component, or 5-component, or 10-component prediction.

49. A method according to claim 48, wherein a 3-component output comprises the prediction for a helix (H), an extended strand (E) and a coil (C).

50. A method according to claim 25, wherein the output of one level of neural networks comprises a descriptor of 2, 3, 4, 5, 6, 7, 8 or 9 consecutive residues, preferably 3, 5, 7, or 9 consecutive residues.

51. A method according to claim 25, wherein the number of neural networks in the one of the subsequent level or levels range from 1 to 1 000 000, such as from 1 to 100 000, 1 to 50 000, 1 to 10 000, 1 to 5000, 1 to 2500, 1 to 1000, 1 to 500, 1 to 250, 1 to 100, 1 to 50, 1 to 25 or 1 to 10.

52. A method of predicting a set of features of an input data by providing said input data to at least 16 diverse neural networks thereby providing an individual prediction of the said set of features on the basis of a weighted average said weighted average comprising an evaluation of the estimation of the prediction accuracy for a protein chain by a prediction means.

53. A method according to claim 52, wherein the estimation of the prediction accuracy is made by summing the per-residue maximum of H versus E versus C probabilities for said protein chain and dividing by the number of amino-acid residues in the protein chain, and
wherein the mean and standard deviation of the accuracy estimation is taken for all prediction means for the protein chain, and
wherein a weighted average is made for substantially all or optionally a subset of prediction means,
wherein the subset comprises those prediction means with estimated accuracy above a threshold consisting of the mean estimated accuracy, the mean accuracy plus one standard deviation above the mean accuracy, or the mean estimated accuracy plus two standard deviations above the mean, or
wherein the subset comprises at least 10 prediction means in cases where the accuracy of fewer than 10 estimated prediction fail to satisfy the threshold,

54. A method according to claim 52, wherein the weighted average comprise a multiplication of each component of a single- or multi-component output for each residue,
said output produced by the selected prediction means by the per-chain estimated accuracy obtained for said chain and prediction means,
and the resulting said products summed by residue and component,
and the resulting sums being divided by the sum of weights,
and the resulting maximal per-residue component quotient being used to determine the H or E or C secondary structure assignment for that residue, and
the per-chain per-prediction probability in the H versus E versus C assignment is averaged over a given protein chain.

55. A method according to claim 52, wherein the set of features comprise secondary structure class assignment, tertiary structure, interatomic distance, bond strength, bond angle, descriptors relating to or reflecting hydrophobicity, hydrophilicity, acidity, basicity, relative nucleophilicity, relative electrophilicity, electron density or rotational freedom, scalar products of atomic vectors, cross products of atomic vectors, angles between atomic vectors, triple scalar products between atomic vectors, torsion angles, atomic angles such as but not exclusively omega, psi, phi, chi1, chi2, chi21, chi3, chi4, chi5 angles, chain curvature, chain torsion angles, torsion vectors and mathematical functions thereof.

56. A method according to claim 52, wherein the input data is provided to at least 20 diverse neural networks, such as at least 30, 40, 50, 60, 70, 80, 90, 100, 200, 500, 1000, 5000, 10 000, 100 000, and 1 000 000.

57. A method of predicting a set of features of input data using outputexpansion wherein a process by which a single- or multi-component output is represented by a descriptor of 2 or more consecutive elements of a sequence, such as residues of a protein sequence.

58. A method for predicting a set of chemical, physical or biological features related to chemical substances or related to interactions of chemical substances using a system comprising a prediction means comprising output expansion,
the method comprising
using at least 1 individual prediction means predicting substantially the whole set of features at least twice thereby providing at least two individual predictions of substantially all of the set of features, and
predicting the set of features either on the basis of combining at least two of the individual predictions, the combining being performed in such a manner that the combined prediction is more accurate on a test set than substantially any of the at least two of the predictions, or
on the basis of selecting one of the sets of predictions, the selection being performed in such a manner that the selected prediction is more accurate on a test set than a prediction from corresponding prediction means without the use of output expansion,
or predicting the set of features on the basis of at least one individual predictions, or
on the basis of combining at least two of the individual predictions, the combining being performed in such a manner that the combined prediction is more accurate on a test set than substantially any of the predictions of the individual prediction means, or more accurate than corresponding prediction means not comprising output expansion.
